(19) United States
(12) Patent Application Publication
(10) Pub. No.: US 2009/0111782 A1
(43) Pub. Date: Apr. 30, 2009

(54) COMPOSITION AND METHODS RELATING TO GLUCOCORTICOID RECEPTOR-ALPHA AND PEROXISOME PROLIFERATOR-ACTIVATED RECEPTORS

(75) Inventors: Karolien De Bosscher, Brakel (BE); Guy Haegeman, Balegem (BE); Nadia Bougarne, Sint-Niklaas (BE)

Correspondence Address: GIFFORD, KRASS, SPRINKLE,ANDERSON & CITKOWSKI, P.C, PO BOX 7021, TROY, MI, 48007-7021, US

(73) Assignee: Ghent University, Ghent (BE)

(21) Appl. No.: 12/252,894

(22) Filed: Oct. 16, 2008

Related U.S. Application Data
(60) Provisional application No. 60/999,119, filed on Oct. 16, 2007.

(51) Int. Cl. A61K 31/56; A61K 31/195; A61K 31/44; A61K 31/426

(52) U.S. Cl. 514/171; 514/563; 514/356; 514/369

(57) ABSTRACT

Methods of treating a glucocorticoid-responsive condition in a subject are provided according to embodiments of the present invention which include administering, in combination, a glucocorticoid receptor agonist and a PPAR agonist in therapeutically effective amounts. It is an aspect of the present invention that the amount of the glucocorticoid receptor agonist used in a method of treating a glucocorticoid-responsive condition is less than an amount of the glucocorticoid receptor agonist necessary to achieve a therapeutic effect if administered in the absence of the PPAR agonist.

### REFERENCE TO RELATED APPLICATION

This application claims priority from U.S. Provisional Patent Application Ser. No. 60/999,119, filed Oct. 16, 2007, the entire content of which is incorporated herein by reference.

### FIELD OF THE INVENTION

Methods and compositions according to embodiments of the present invention relate generally to treatment of glucocorticoid-responsive conditions and reduction and prevention of glucocortioid-induced side-effects in a subject. In particular embodiments of the present invention, compositions are described which include one or more PPAR agonists for administration to a subject to reduce and prevent glucocortioid-induced side-effects in the subject.

### BACKGROUND OF THE INVENTION

Glucocorticoids (GCs) are used for the treatment of acute and chronic inflammatory diseases. GCs mediate their effect via the Glucocorticoid Receptor (GR) (Hollenberg and Evans, 1988; Wright et al., 1993), a member of the nuclear steroid/thyroid hormone receptor superfamily (Beato et al., 1995; Mangelsdorf et al., 1995; Robinson-Rechavi et al., 2003). The inactive GR usually resides in the cytoplasm of the cell in a complex with chaperoning proteins (Pratt et al., 2006). After binding of GCs to the receptor, a conformational change in the receptor is induced, releasing the chaperoning proteins and allowing GR to translocate into to the nucleus. Activated GR can directly regulate the expression of its target genes through binding as a homodimer onto GREs, located in the promoter region. Target genes of GRα include proteins involved in glucose (glc), fat and protein metabolism. In addition, GRα can also influence gene expression by interfering with the activity of Nuclear Factor-kappa B (NF-κB), a key regulatory pro-inflammatory transcription factor (De Bosscher et al., 2006).

At present, glucocorticoids are among the most potent drugs for the treatment of acute and chronic inflammatory diseases. However, side effects, such as osteoporosis, muscle wasting, hypertension, behavioral alterations, and disorders of glucose and lipid metabolism, burdens their therapeutic use (Boumpas et al., 1993; Rosen and Miner, 2005).

There is a continuing need for compositions and methods for treating glucocorticoid-responsive conditions and for reducing glucocorticoid side-effects.

### SUMMARY OF THE INVENTION

Methods of treating a glucocorticoid-responsive condition in a subject are provided according to embodiments of the present invention which includes administering, in combination, a glucocorticoid receptor agonist and at least one PPAR agonist in therapeutically effective amounts.

In particular embodiments, a method of treating a glucocorticoid-responsive condition in a subject, is provided which includes administering, in combination, a glucocorticoid receptor agonist and a PPARα agonist, a PPARγ agonist, a PPARδ agonist, a dual PPARα/γ agonist, a pan PPAR agonist or a combination of any two or more of a PPARα agonist, a PPARγ agonist, a PPARδ agonist, a dual PPARα/γ agonist and a pan PPAR agonist, in therapeutically effective amounts.

Fibrates are PPARα agonists which can be included in compositions and methods of the present invention.

Examples of PPARα agonists which can be included in compositions and methods of the present invention include beclofibrate, bezafibrate, ciprofibrate, clofibrate, etofibrate, fenofibrate, gemfibrozil, 2-methyl-2-(4-((4-methyl-2-(4-(trifluoromethyl)phenyl)thiazole-5-carboxamido)methyl)phenoxy)propanoic acid; 2-methyl-2-[[4-[2-[[(cyclohexylamino)carbonyl](4-cyclohexylbutyl)amino]ethyl]phenyl]thio]-propanoic acid; 2-[[4-[2-[[[(2,4-difluorophenyl)amino]carbonyl]heptylamino]ethyl]phenyl]thio]-2-methyl-propanoic acid; [[4-chloro-6-[(2,3-dimethylphenyl)amino]-2-pyrimidinyl]thio]-acetic acid; 2-methyl-2-(4-{3-[1-(4-methylbenzyl)-5-oxo-4,5-dihydro-1H-1,2,4-triazol-3-yl]propyl}phenoxy)propanoic acid (LY518674); and [2-(4(2-(1-Cyclohexanebutyl-3-cyclohexylureido)ethyl)phenylthio)-2-methylpropionic acid, also known as GW7647 and referred to herein as GW647.

Examples of glucocorticoid receptor agonists which can be included in compositions and methods of the present invention include alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, medrysone, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide and ulobetasol.

It is an aspect of the present invention that the amount of the glucocorticoid receptor agonist used in a method of treating a glucocorticoid-responsive condition is less than an amount of the glucocorticoid receptor agonist necessary to achieve a therapeutic effect if administered in the absence of the PPAR agonist.

It is an aspect of the present invention that administration of a PPARα agonist, a PPARγ agonist, a PPARδ agonist, a dual PPARα/γ agonist and/or a pan PPAR agonist, reduces side-effects of administration of glucocorticoid receptor agonists.

Compositions are provided according to embodiments of the present invention which include a glucocorticoid receptor agonist, at least one PPAR agonist and a pharmaceutically acceptable carrier. In preferred compositions, a glucocorticoid receptor agonist and a PPAR agonist selected from a PPARα agonist, a PPARγ agonist, a PPARδ agonist, a dual PPARα/γ agonist a pan PPAR agonist or a combination of any two or more PPAR agonists, are each present in an amount which, in combination, is a therapeutically effective amount for treating a glucocorticoid-responsive condition in a subject. Particular compositions include a glucocorticoid receptor agonist, a PPARα agonist and a pharmaceutically acceptable carrier.

In particular embodiments of inventive compositions, the amount of the glucocorticoid receptor agonist is less than an amount of the glucocorticoid receptor agonist necessary to achieve a therapeutic effect if administered without the PPAR agonist.

Compositions according to embodiments of the present invention include an amount of a PPAR agonist sufficient to reduce a side-effect of administration of a glucocorticoid receptor agonist.

Kits according to embodiments of the present invention include a glucocorticoid receptor agonist, a PPAR agonist, or both a glucocorticoid receptor agonist and a PPAR agonist. Kits can include a composition including both a glucocorticoid receptor agonist and a PPAR agonist. Instructions for administering a glucocorticoid receptor agonist and a PPAR agonist for treatment of a glucocorticoid-responsive condition in a subject are included in preferred embodiments of an inventive kit. A PPAR agonist included in a kit of the present invention can be a PPARα agonist, a PPARγ agonist, a PPARδ agonist, a dual PPARα/γ agonist a pan PPAR agonist or a combination of any two or more PPAR agonists. In particular embodiments, the PPAR agonist is a PPARα agonist.

Methods of treating insulin resistance in a subject are provided according to embodiments of the present invention which include administering, in combination, a glucocorticoid receptor agonist and a PPAR agonist in therapeutically effective amounts. In particular embodiments, the glucocorticoid receptor agonist is administered prior to the PPAR agonist. Optionally, the glucocorticoid receptor agonist is administered substantially simultaneously with the PPAR agonist. A PPAR agonist administered according to embodiments of methods of treating insulin resistance of the present invention can be a PPARα agonist, a PPARγ agonist, a PPARδ agonist, a dual PPARα/γ agonist a pan PPAR agonist or a combination of any two or more PPAR agonists. In particular embodiments, the PPAR agonist is a PPARα agonist. Beneficial effects of such treatment include an increase in insulin sensitivity as measured by any of various standard methods.

### BRIEF DESCRIPTION OF THE DRAWINGS

FIG. 1A is a graph showing the effects of GRα agonists and/or Peroxisome Proliferator-Activated Receptor α (PPARα) agonists on TNF-induced IL-6 production;

FIG. 2A is an image of PCR products showing the effects of GRα agonists and/or Peroxisome Proliferator-Activated Receptor α (PPARα) agonists on mRNA levels of human placental alkaline phosphatase (hPAP) compared to a loading control, GAPDH;

FIG. 2B is a graph showing the effects of GRα agonists and/or PPARα agonists on glucocorticoid-induced leucine zipper (GILZ) mRNA levels;

FIG. 2C is a graph showing the effects of GRα agonists and/or PPARα agonists on glucose-6-phosphatase (G6 Pase) mRNA levels;

FIG. 2D is a graph showing the effects of GRα agonists and/or PPARα agonists on luciferase expression from an expression construct including a glucocorticoid response element, in the presence or absence of exogenously expressed PPARα, as measured by luciferase enzyme activity;

FIG. 3A is a graph showing the effects of PPARα agonists on induction of a PPARα-induced gene, PDK-4, in wild-type mice and PPARα knockout mice;

FIG. 3B is a graph showing the effects of GRα agonists and/or PPARα agonists on GILZ mRNA levels in wild-type mice and PPARα knockout mice;

FIG. 3C is a graph showing the effects of GRα agonists and/or PPARα agonists on SGK mRNA levels in wild-type mice and PPARα knockout mice;

FIG. 4A is a graph showing the effects of GRα agonists and/or PPARα agonists on GILZ mRNA levels in mice;

FIG. 4B is a graph showing the effects of GRα agonists and/or PPARα agonists on ACO mRNA levels in mice;

FIG. 5 is a graph showing the effects of GRα agonists and/or PPARα agonists on blood glucose levels in high-fat diet fed and insulin-resistant mice;

FIG. 6A is an image of immunoblots showing the effects of GRα agonists and/or PPARα agonists on the subcellular localization of GRα;

FIG. 6B is an image of immunoblots showing ligand-independent physical interaction GRα and PPARα;

FIG. 7A is a graph showing the effects of GRα agonists and/or PPARα agonists on recruitment of GRα to a promoter glucocorticoid response element (GRE);

FIG. 7B is a graph showing the effects of GRα agonists and/or PPARα agonists on recruitment of RNA pol II to a promoter;

FIG. 8A is a graph showing the effects of GRα agonists and/or PPARα agonists on TNF-induced IL-6 production;

FIG. 8B is a graph showing the effects of GRα agonists and/or PPARα agonists on TNF-induced MCP-1 mRNA levels using quantitative RT-PCR analysis;

FIG. 8C is a graph showing the effects of GRα agonists and/or PPARα agonists on TNF-induced MMP9 mRNA levels using quantitative RT-PCR analysis;

FIG. 9A is a graph showing the effects of GRα agonists and/or PPARα agonists on liver weights of treated mice;

FIG. 9B is a graph showing the effects of GRα agonists and/or PPARα agonists on thymus weights of treated mice;

FIG. 10A is an image of an immunoblot showing GST-pull down of endogenous proteins;

FIG. 10B is an image of an immunoblot showing immunoprecipitation assays of endogenous proteins of endogenous proteins;

FIG. 11A is a graph showing the effects of GRα agonists and/or PPARγ agonists on luciferase expression from an expression construct as measured by luciferase enzyme activity; and

FIG. 11B is a graph showing the effects of GRα agonists and/or PPARγ agonists on reporter expression from an expression construct.

### DETAILED DESCRIPTION OF THE INVENTION

Compositions and methods for treating glucocorticoid-responsive conditions and for reducing and preventing side-effects of glucocorticoid treatment in a subject are provided by the present invention.

Methods of treating a glucocorticoid-responsive condition in a subject are provided according to embodiments of the present invention which includes administering, in combination, a glucocorticoid receptor agonist and a PPAR agonist in therapeutically effective amounts.

In particular embodiments, a method of treating a glucocorticoid-responsive condition in a subject, is provided which includes administering, in combination, a therapeutically effective amount of a glucocorticoid receptor agonist and a therapeutically effective amount of a PPARα agonist, a PPARγ agonist, a PPARδ agonist, a dual PPARα/γ agonist and/or a pan PPAR agonist.

Methods of treating a glucocorticoid-responsive condition in a subject are provided according to embodiments of the present invention which include administering in combination, a glucocorticoid receptor agonist and a PPARα agonist in therapeutically effective amounts.

The phrase “administering in combination” as used herein refers to any form of administration of a glucocorticoid receptor agonist and one or more PPAR agonists such that the PPAR agonist is administered to a subject while a previously administered glucocorticoid receptor agonist is still effective in the subject or such that the glucocorticoid receptor agonist is administered to a subject while a previously administered PPAR agonist is still effective in the subject.

The terms “treating” and “treatment” used to refer to treatment of a glucocorticoid-responsive condition in a subject includes: preventing, inhibiting or ameliorating the glucocorticoid-responsive condition in a subject, such as slowing progression of the condition and/or reducing or ameliorating a sign or symptom of the condition; and preventing, inhibiting or ameliorating a side-effect of glucocorticoid administration glucocorticoid-responsive condition in a subject. The terms “treating” and “treatment” are also used herein to refer to treatment of insulin resistance in a subject, such as glucocorticoid-induced insulin resistance and insulin resistance resulting from factors such as high fat content diet, and include preventing, inhibiting or ameliorating insulin resistance in a subject.

Treatment of a glucocorticoid-responsive condition with a combination of a glucocorticoid receptor agonist and at least one PPAR agonist selected from a PPARα agonist, a PPARγ agonist, a PPARδ agonist, a dual PPARα/γ agonist, a pan PPAR agonist and a combination of two or more PPAR agonists allows for use of lower dosages of both the glucocorticoid receptor agonist and the PPAR agonist to achieve a therapeutic effect than when either agonist is used alone. Thus, it is an aspect of the present invention that the amount of the glucocorticoid receptor agonist used in a method of treating a glucocorticoid-responsive condition is less than an amount of the glucocorticoid receptor agonist necessary to achieve a therapeutic effect if administered in the absence of the PPAR agonist or combination of PPAR agonists.

In embodiments of the present invention, treatment of a glucocorticoid-responsive condition with a combination of a glucocorticoid receptor agonist and a PPARα agonist allows for use of lower dosages of both the glucocorticoid receptor agonist and the PPARα agonist to achieve a therapeutic effect than when either agonist is used alone. Thus, it is an aspect of the present invention that the amount of the glucocorticoid receptor agonist used in a method of treating a glucocorticoid-responsive condition is less than an amount of the glucocorticoid receptor agonist necessary to achieve a therapeutic effect if administered in the absence of the PPARα agonist.

In particular embodiments of the present invention, the amount of the glucocorticoid receptor agonist administered is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90%, less than an amount of the glucocorticoid receptor agonist necessary to achieve a therapeutic effect if administered without the PPARα agonist, PPARγ agonist, PPARδ agonist, dual PPARα/γ agonist, pan PPAR agonist or combination of PPAR agonists. The amount of the glucocorticoid receptor agonist administered can be less than 5% or more than 90%, less than an amount of the glucocorticoid receptor agonist necessary to achieve a therapeutic effect if administered without the PPARα agonist, PPARγ agonist, PPARδ agonist, dual PPARα/γ agonist, pan PPAR agonist or combination of PPAR agonists.

Side effects of glucocorticoid treatment can be bothersome or even crippling. Side-effects of glucocorticoid receptor agonists include osteoporosis, glaucoma, hyperglycemia, diabetes mellitus, sodium retention, hypertension, edematous face and other tissues, increased susceptibility to infection, decreased rate of wound healing, cataracts, acne, myopathy, thinning of the skin, redistribution of body fat to the nape of the neck and lower abdomen, suppression of the hypothalamic-pituitary-adrenal axis, euphoria, depression, psychoses, anorexia, colonic ulceration, and hyperlipidemia.

Methods of the present invention include administration of at least one PPAR agonist to prevent one or more glucocorticoid receptor agonist side-effects. It is a surprising aspect of methods of treatment of the present invention that administration of a glucocorticoid receptor agonist and at least one PPAR agonist in combination for treatment of a glucocorticoid-responsive condition reduces or prevents glucocorticoid receptor agonist side-effects. In particular embodiments, administration of one or more PPARα agonists reduces or prevents one or more glucocorticoid receptor agonist side-effects.

In particular embodiments of the present invention, a PPAR agonist is administered to prevent or reduce hyperglycemia in a subject to whom a glucocorticoid receptor agonist has been or will be administered.

In embodiments of methods of the present invention, a glucocorticoid receptor agonist and a PPAR agonist are administered, in combination, to a subject having insulin resistance. Surprisingly, combined administration of a glucocorticoid receptor agonist and a PPAR agonist prevents or reduces glucocorticoid-induced side-effects such as hyperglycemia. Such methods are useful, for instance, in treating an insulin-resistant subject

In particular embodiments of the present invention, a glucocorticoid receptor agonist and at least one PPAR agonist are administered substantially simultaneously to a subject having insulin resistance. In certain embodiments of the present invention, at least one PPAR agonist is administered to a subject having insulin resistance after administration of a glucocorticoid receptor agonist to the subject.

In particular embodiments of the present invention, a PPARα agonist, PPARγ agonist, a PPARδ agonist, dual PPARα/γ agonist, pan PPAR agonist or combination of PPAR agonists is administered to prevent or reduce insulin resistance in a subject to whom a glucocorticoid receptor agonist has been or will be administered. In a particular example, a PPARα agonist and a glucocorticoid receptor agonist are administered in combination to prevent or reduce insulin resistance in a subject.

Methods of the present invention include administration of a PPARα agonist, a PPARγ agonist, a PPARδ agonist, a dual PPARα/γ agonist, a pan PPAR agonist or combination of PPAR agonists agonist to prevent one or more glucocorticoid receptor agonist side-effects.

In particular embodiments of the present invention, a PPARγ agonist is administered to prevent or reduce hyperglycemia in a subject to whom a glucocorticoid receptor agonist has been or will be administered.

In particular embodiments of the present invention, a PPARγ agonist is administered to prevent or reduce insulin resistance in a subject to whom a glucocorticoid receptor agonist has been or will be administered. In a particular example, a PPARγ agonist and a glucocorticoid receptor agonist are administered in combination to prevent or reduce insulin resistance in a subject.

In particular embodiments of the present invention, both a PPARα agonist and/or a PPARγ agonist are administered to prevent or reduce insulin resistance in a subject to whom a glucocorticoid receptor agonist has been or will be administered. In a particular example, a PPARα agonist, a PPARγ agonist and a glucocorticoid receptor agonist are administered in combination to prevent or reduce insulin resistance in a subject.

Methods of the present invention include administration of a PPARα agonist and/or a PPARγ agonist to prevent one or more glucocorticoid receptor agonist side-effects.

In particular embodiments of the present invention, a PPARα agonist and/or a PPARγ agonist are administered to prevent or reduce hyperglycemia in a subject to whom a glucocorticoid receptor agonist has been or will be administered.

In particular embodiments of the present invention, a PPARα agonist and/or a PPARγ agonist are administered to prevent or reduce insulin resistance in a subject to whom a glucocorticoid receptor agonist has been or will be administered. In a particular example, a PPARα agonist, a PPARγ agonist and a glucocorticoid receptor agonist are administered in combination to prevent or reduce insulin resistance in a subject.

The term “glucocorticoid receptor agonist” refers to a substance that interacts with a glucocorticoid receptor and enhances or increases a function of the glucocorticoid receptor. The term “glucocorticoid receptor agonist” encompasses both full and partial glucocorticoid receptor agonists. The term “glucocorticoid receptor agonist” encompasses selective modulators of the glucocorticoid receptor (SGRMs). SGRMs are known in the art, for example as described in Elmore, S. W., et al., J. Med. Chem. 44, 4481-4491; H. C. Owen, et al., Mol Cell Endocrinol 264 (2007), pp. 164-170 and De Bosscher K, et al., Proc Natl Acad Sci USA. 2005 Nov. 1; 102(44):15827-32.

Glucocorticoid receptor agonist activity is identified using any of various standard assays such as assays for glucocorticoid receptor binding, assays for transactivation or transrepression of a glucocorticoid-responsive gene, and assays for dissociated ligand effects, for instance as described in Chen, T., Curr. Opin. Chem. Biol., 12:418-426, 2008.

The term “PPAR agonist” refers to any PPARα agonist, PPARγ agonist, dual PPARα/γ agonist or pan PPAR agonist. PPAR agonist activity is identified using any of various standard assays such as assays for PPARα, PPARγ, and/or PPARδ binding, for transactivation or transrepression of a PPAR-responsive gene and assays for dissociated ligand effects, for instance as described in Chen, T., Curr. Opin. Chem. Biol., 12:418-426, 2008.

The term “PPARα agonist” refers to a substance that interacts with PPARα and enhances or increases a function of PPARα. The term “PPARα agonist” encompasses both full and partial PPARα agonists. PPARα agonist activity is identified using any of various standard assays such as PPARα binding assays and in-vitro transcription assays. The term “PPARα agonist” encompasses selective modulators of the PPARα (SPPARαMs). SPPARαMs are known in the art, for example, as described in Pourcet et al., Expert Opin. Emerging Drugs (2006) 11(3):379-401.

The term “PPARγ agonist” refers to a substance that interacts with PPARγ and enhances or increases a function of PPARγ. The term “PPARγ agonist” encompasses both full and partial PPARγ agonists. PPARγ agonist activity is identified using any of various standard assays such as PPARγ binding assays and in-vitro transcription assays. The term “PPARγ agonist” encompasses selective modulators of the PPARγ (SPPARγMs). SPPARγMs are known in the art and include FK-614; 5-substituted 2-benzoylaminobenzoic acids derivatives: BVT-13, -762, -763; 3-benzoyl derivatives; 3-Benzisoxazoyl derivatives; and PA-082 described in Pourcet et al., Expert Opin. Emerging Drugs (2006) 11(3):379-401.

The term “PPARδ agonist” refers to a substance that interacts with PPARδ and enhances or increases a function of PPARδ. The term “PPARδ agonist” encompasses both full and partial PPARδ agonists. PPARδ agonist activity is identified using any of various standard assays such as PPARδ binding assays and in-vitro transcription assays. The term “PPARδ agonist” encompasses selective modulators of the PPARδ (SPPARδMs). Exemplary PPARδ agonists include GW-610,742 as described in van der Veen J N, et al., J. Lipid Res. 46 (3): 526-34, 2005 and GW501516 as described in Sznaidman M L, et al., Bioorg. Med. Chem. Lett. 13 (9): 1517-21, 2003; and Dimopoulos N, et al., FEBS Lett. 581 (24): 4743-8, 2007.

In certain embodiments of inventive compositions and methods, dual PPARα/PPARγ agonists and/or pan PPAR agonists can be used. Examples of dual PPARα/PPARγ agonists include glitazars and others such as those described in Pourcet et al., Expert Opin. Emerging Drugs (2006) 11(3):379-401. Examples of pan PPAR agonists illustratively include bezafibrate and BPR1H036 and others such as those described in Pourcet et al., Expert Opin. Emerging Drugs (2006) 11(3):379-401.

Fibrates are PPARα agonists optionally included in compositions and methods of the present invention. Fibrates are well-known derivatives of fibric acid, illustratively including but not limited to, beclofibrate, bezafibrate, ciprofibrate, clofibrate, etofibrate, fenofibrate and gemfibrozil.

Examples of PPARα agonists included in compositions and methods of the present invention include, but are not limited to, 2-methyl-2-(4-((4-methyl-2-(4-(trifluoromethyl)phenyl)thiazole-5-carboxamido)methyl)phenoxy)propanoic acid, see J. Med. Chem., 50:685-695, 2007, CAS Reg. No. 622402-22-6; 2-methyl-2-[[4-[2-[[(cyclohexylamino)carbonyl](4-cyclohexylbutyl)amino]ethyl]phenyl]thio]-propanoic acid, see Bioorg. Med. Chem. Lett., 11:1225-1227, 2001, CAS Reg. No. 265129-71-3; 2-[[4-[2-L[[(2,4-difluorophenyl)amino]carbonyl]heptylamino]ethyl]phenyl]thio]-2-methyl-propanoic acid, see J. Biol. Chem., 275:16638-16642, 2000, CAS Reg. No. 247923-29-1; [[4-chloro-6-[(2,3-dimethylphenyl)amino]-2-pyrimidinyl]thio]-acetic acid, also known as WY 14643, CAS Reg. No. 50892-23-4; 2-methyl-2-(4-{3-[1-(4-methylbenzyl)-5-oxo-4,5-dihydro-1H-1,2,4-triazol-3-yl]propyl}phenoxy)propanoic acid (LY518674); and 2-(4-(2-(1-Cyclohexanebutyl-3-cyclohexylureido)ethyl)phenylthio)-2-methylpropionic acid, also known as GW647, see Curr. Opin. Lipidol., 14:459-468, 2003.

Pharmaceutically acceptable salts, solvates and/or prodrugs of PPARα agonists can be used. Combinations of two or more PPARα agonists are contemplated as within the scope of the present invention.

Non-limiting examples of naturally occurring and synthetic glucocorticoid receptor agonists which can be included in compositions and methods of the present invention are alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, medrysone, meprednisone, 6a-methylprednisolone, methylprednisolone, methylprednisolone acetate, methylprednisolone aceponate, mometasone, mometasone furoate, mometasone Furoate monohydrate, paramethasone, prednicarbate, prednisolone, prednisone, prednylidene, rimexolone, tixocortol, triamcinolone, triamcinolone acetonide and ulobetasol. Pharmaceutically acceptable salts, solvates and/or prodrugs of glucocorticoid receptor agonists can be used. Combinations of two or more glucocorticoid receptor agonists are contemplated as within the scope of the present invention.

Examples of PPARγ agonists included in compositions and methods of the present invention include, but are not limited to, thiazolidinediones (TZDs) such as rosiglitazone, pioglitazone, rivoglitazone and troglitazone.

The terms “pharmaceutically acceptable salt,” “pharmaceutically acceptable solvate” and “pharmaceutically acceptable prodrug” refers to salts, solvates and/or prodrugs which are suitable for use in a subject without undue toxicity or irritation to the subject and which are effective for their intended use.

Pharmaceutically acceptable salts include pharmaceutically acceptable acid addition salts and base addition salts. Pharmaceutically acceptable salts are well-known in the art, such as those detailed in S. M. Berge et al., J. Pharm. Sci., 66:1-19, 1977. Exemplary pharmaceutically acceptable salts are those suitable for use in a subject without undue toxicity or irritation to the subject and which are effective for their intended use which are formed with inorganic acids such as hydrochloric acid, hydrobromic acid, hydroiodic acid, nitric acid, phosphoric acid, sulfuric acid and sulfamic acid; organic acids such as acetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, formic acid, fumaric acid, glutamic acid, glycolic acid, glycerophosphoric acid, hemisulfic acid, heptanoic acid, hexanoic acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, trichloroacetic acid, trifluoroacetic acid and undecanoic acid; inorganic bases such as ammonia, hydroxide, carbonate, and bicarbonate of ammonium; organic bases such as Primary, secondary, tertiary and quaternary amine compounds ammonium, arginine, betaine, choline, caffeine, diolamine, diethylamine, diethanolamine, 2-dimethylaminoethanol, 2-diethylaminoethanol, dicyclohexylamine, dicyclohexylamine, dibenzylamine, N,N-dibenzylphenethylamine, 1-ephenamine, N,N′-dibenzylethylenediamine, ethanolamine, ethylamine, ethylenediamine, glucosamine, histidine, hydrabamine, isopropylamine, 1 h-imidazole, lysine, methylamine, N-ethylpiperidine, N-methylpiperidine, N-methylmorpholine, N,N-dimethylaniline, piperazine, trolamine, methylglucamine, purines, piperidine, pyridine, theobromine, tetramethylammonium compounds, tetraethylammonium compounds, trimethylamine, triethylamine, tripropylamine and tributylamine and metal cations such as aluminum, calcium, copper, iron, lithium, magnesium, manganese, potassium, sodium, and zinc.

Solvates illustratively include hydrates, ethanolates, methanolates.

Synthesis of glucocorticoid receptor agonists and PPAR agonists is well known. Particular examples are described in R. Vardanyan and V. Hruby, Synthesis of Essential Drugs, Elsevier Science, 2006.

A subject treated according to methods and using compositions of the present invention can be mammalian or non-mammalian. A mammalian subject can be any mammal including, but not limited to, a human; a non-human primate; a rodent such as a mouse, rat, or guinea pig; a domesticated pet such as a cat or dog; a horse, cow, pig, sheep, goat, or rabbit. A non-mammalian subject can be any non-mammal including, but not limited to, a bird such as a duck, goose, chicken, or turkey.

The term “glucocorticoid-responsive condition” refers to any disease or condition for which administration of one or more glucocorticoids has a beneficial effect. Glucocorticoid-responsive conditions that can be treated using compositions and methods of the present invention include, but are not limited to, inflammatory conditions and proliferative disorders.

Glucocorticoid-responsive conditions are well-known and include glucocorticoid-responsive systemic and localized conditions such as glucocorticoid-responsive conditions involving the upper airway passages, lower airway passages and/or lungs; skin; musculo-skeletal system including bones, joints, connective tissue and muscle; gastrointestinal system including esophagus, intestines, mouth, salivary glands, stomach, liver, gallbladder, pancreas, rectum, and anus; circulatory system including blood vessels and heart; lymphatic system including lymph vessels and nodes; endocrine system; urinary system including kidneys, bladder, urethra and ureters; central and/or peripheral nervous system; and sensory organs.

Exemplary glucocorticoid-responsive conditions involving the upper airway passages, lower airway passages and/or lungs are adult respiratory distress syndrome, bronchiectasis, bronchial asthma, bronchitis, cystic fibrosis, pulmonary fibrosis, pulmonary inflammation, chronic obstructive pulmonary disease, edema, granulomatosis and sarcoidosis.

Exemplary glucocorticoid-responsive conditions involving the skin are acne vulgaris, acne rosacea conglobata, acne rosacea fulminans, allergic uticaria, atopic dermatitis, eczema, psoriasis, pityriasis rubra pilaris, erythematous conditions, bullous dermatoses, epidermolysis bullosa, icthyoses, lichen planus, lichen simplex chronicus, lichenoid purpura, lichen sclerosus, pruritus, seborrheic dermatitis, rosacea, pemphigus vulgaris, erythema multiforme exudativum; alopecia areata, alopecia totalis, scarring, keloids, cutaneous sarcoidosis, pemphigoid gestationis, pemphigus vulgaris, wounds, burns, blisters, and cutaneous T cell lymphomas.

Exemplary glucocorticoid-responsive conditions involving the musculo-skeletal system such as bones, joints, connective tissue and/or muscle are dermatomyositis, arthritic conditions generally, idiopathic arthritis; rheumatic diseases such as rheumatoid arthritis, juvenile rheumatoid arthritis; acute rheumatic fever, and polymyalgia rheumatica; rheumatoid spondylitis, gouty arthritis, osteoarthritis, polymyositis, systemic lupus erythematosus, scleroderma, Sjogren syndrome and Still disease.

Exemplary glucocorticoid-responsive conditions involving the gastrointestinal system are biliary atresia, cirrhosis, Crohn's disease, distal proctitis, gastritis, gastroenteritis, hemorrhoids, hepatitis, idiopathic proctitis, inflammatory bowel disease, sclerosing cholangitis and ulcerative colitis.

Exemplary glucocorticoid-responsive conditions involving the circulatory system are atherosclerosis, Churg-Strauss syndrome, giant cell arteritis, Kawasaki disease, hypersensitivity vasculitis, mycocarditis, microscopic polyangiitis, polyarteritis nodosa, rheumatic carditis, Takayasu's arteritis, vasculitis and Wegener's granulomatosis.

Exemplary glucocorticoid-responsive conditions involving the lymphatic system are histiocytic necrotizing lymphadenitis and proliferative diseases involving lymph nodes.

Exemplary glucocorticoid-responsive conditions involving the endocrine system are thyroiditis; and deficiencies such as Addison's disease and adrenocortical insufficiency.

Exemplary glucocorticoid-responsive conditions involving the urinary system are lupus neptritis, nephrotic syndrome, post-obstructive syndrome, tubular ischemia, and nephritis such as glomerulonephritis.

Exemplary glucocorticoid-responsive conditions involving the nervous system are Bell's palsy, edema and multiple sclerosis.

Exemplary glucocorticoid-responsive conditions involving the sensory organs are chorioretinitis, conjunctivitis, iritis, keratoconjunctivitis sicca, scleritis, uveitis, and macular edema.

Glucocorticoid-responsive inflammatory conditions are well-known and include systemic inflammatory conditions as well as organ, tissue or system-specific inflammatory conditions. For example, glucocorticoid-responsive inflammatory conditions include inflammatory conditions of the respiratory system such as inflammatory conditions of the upper airway passages, lower airway passages and/or lungs; inflammatory conditions of the skin; musculo-skeletal system including bones, joints, connective tissue and muscle; gastrointestinal system including esophagus, intestines, mouth, salivary glands, stomach, liver, gallbladder, pancreas, rectum, and anus; circulatory system including blood vessels and heart; lymphatic system including lymph vessels and nodes; endocrine system; urinary system including kidneys, bladder, urethra and ureters; central and/or peripheral nervous system; and sensory organs.

Non-limiting examples glucocorticoid-responsive inflammatory conditions which can be treated using compositions and methods of the present invention include: acne vulgaris; acne rosacea conglobata; acne rosacea fulminans; acute febrile neutrophilic dermatosis; acute respiratory distress syndrome; adrenogenital syndrome; allergic reaction; allergic conjunctivitis; allergic rhinitis; allergic intraocular inflammatory diseases; allergic uticaria; anaphylactic reaction; ANCA-associated small-vessel vasculitis; angioedema; ankylosing spondylitis; aphthous stomatitis; arthritis; atherosclerosis; atopic dermatitis; Behcet's disease; Bell's palsy; berylliosis; bronchial asthma; bullous herpetiformis dermatitis; bullous pemphigoid; bursitis; carditis; celiac disease; cerebral ischaemia; chorioretinitis; chronic obstructive pulmonary disease; cirrhosis; Cogan's syndrome; contact dermatitis; Crohn's disease; cutaneous lesions of systemic lupus erythematosus; cutaneous sarcoidosis; dermatitis; dermatomyositis; discoid lupus erythematosus; eosinophilic fasciitis; epicondylitis; erythema nodosum; exfoliative dermatitis; fibromyalgia; focal glomerulosclerosis; giant cell arteritis; gout; gouty arthritis; graft-versus-host disease; Henoch-Schonlein purpura; herpes gestationis; hirsutism; hypersensitivity drug reactions; idiopathic arthritis; idiopathic cerato-scleritis; idiopathic pulmonary fibrosis; idiopathic thrombocytopenic purpura; inflammation-associated pain; inflammation secondary to trauma; inflammatory bowel or gastrointestinal disorders; inflammatory dermatoses; inflammatory musculoskeletal and connective tissue disorders; juvenile rheumatoid arthritis; laryngeal edema; lichen planus; lichen simplex chronicus; Loeffler's syndrome; lupus nephritis; lupus vulgaris; lymphomatous tracheobronchitis; macular edema; multiple sclerosis; myasthenia gravis; myocarditis; myositis; obstructive pulmonary disease; ocular inflammation; osteoarthritis; pancreatitis; pemphigoid gestationis; pemphigus vulgaris; periodontal disease, polyarteritis nodosa; polymyalgia rheumatica; primary biliary cirrhosis; pruritus; psoriasis; psoriatic arthritis; Reiter's disease; relapsing polychondritis; rheumatic carditis; rheumatic fever; rheumatoid arthritis; sarcoidosis; scleroderma; segmental glomerulosclerosis; septic shock; serum sickness; Sjogren's syndrome; Still's disease; systemic dermatomyositis; systemic lupus erythematosus; Takayasu's arteritis; tendinitis; thyroiditis; ulcerative colitis; uveitis; vasculitis; and Wegener's granulomatosis.

Glucocorticoid-responsive inflammatory conditions include autoimmune diseases such as rheumatoid arthritis, systemic lupus erythematosus, autoimmune hemolytic anemia; autoimmune hepatitis; Guillain-Barré syndrome and inflammatory bowel disease.

Glucocorticoid-responsive proliferative conditions illustratively include acute lymphatic leukemia; chronic lymphocytic leukemia; malignant lymphoma; lymphogranulomatosis; lymphosarcoma; and multiple myeloma.

Glucocorticoid-responsive conditions include tissue and organ transplantation and graft-versus-host disease. Glucocorticoid-responsive conditions include blood disorders illustratively including acquired hemolytic anemia; non-hemolytic anemia, granulocytopenia, and idiopathic thrombocytopenia. Glucocorticoid-responsive conditions include deficiencies such as Addison's disease and adrenocortical insufficiency.

Compositions and methods of the present invention are applicable to any condition having an inflammatory component and are not intended to be limited to use in conditions described herein.

For use in methods of the present invention, a glucocorticoid receptor agonist and/or at least one PPAR agonist can be administered per se or with a pharmaceutically acceptable carrier.

Embodiments of methods of the present invention include administration of a glucocorticoid receptor agonist and at least one PPAR agonist at various times relative to each other, so long as the at least one PPAR agonist is administered to a subject while a previously administered glucocorticoid receptor agonist is still effective in the subject or such that the glucocorticoid receptor agonist is administered to a subject while a previously administered PPAR agonist is still effective in the subject.

Embodiments of methods of the present invention include administration of a glucocorticoid receptor agonist and at least one PPAR agonist at various times relative to each other, so long as the at least one PPAR agonist is administered to a subject while a previously administered glucocorticoid receptor agonist is still effective in the subject or such that the glucocorticoid receptor agonist is administered to a subject while a previously administered PPAR agonist is still effective in the subject.

In particular embodiments of methods of the present invention, a glucocorticoid receptor agonist and at least one PPAR agonist are administered to a subject substantially simultaneously, for instance, in the form of a composition containing both agonists. Alternatively, a glucocorticoid receptor agonist and at least one PPAR agonist are administered to a subject substantially simultaneously in the form of a first composition containing the glucocorticoid receptor agonist and a second composition containing the at least one PPAR agonist, where the first and second compositions are administered to the subject within less than about one hour of each other.

In particular embodiments of methods of the present invention, a glucocorticoid receptor agonist and a PPARα agonist are administered to a subject substantially simultaneously, for instance, in the form of a composition containing both agonists. Alternatively, a glucocorticoid receptor agonist and a PPARα agonist are administered to a subject substantially simultaneously in the form of a first composition containing the glucocorticoid receptor agonist and a second composition containing the PPARα agonist, where the first and second compositions are administered to the subject within less than about one hour of each other.

A “therapeutically effective amount” refers to an amount effective to achieve a desired therapeutic effect, particularly prevention or amelioration of signs or symptoms of a glucocorticoid-responsive condition and/or prevention or amelioration of one or more side effects of glucocorticoid treatment.

Glucocorticoid receptor agonist dosage is typically expressed in terms of “prednisone equivalents.” The number or fraction of “prednisone equivalents” in a given dose of a particular glucocorticoid receptor agonist is generally known in the art or can be determined using conventional pharmacological assays.

In some embodiments, a low dose of a glucocorticoid receptor agonist is administered. A low dosage of a glucocorticoid receptor agonist is less than or equal to 7.5 mg prednisone equivalent per day, see F. Buttgereit et al., Ann. Rheum. Dis., 61:718-722, 2002. A medium dosage of a glucocorticoid receptor agonist is greater than 7.5 mg and less than or equal to 30 mg prednisone equivalent per day. A high dosage of a glucocorticoid receptor agonist is greater than 30 mg and less than or equal to 100 mg prednisone equivalent per day, while a very high dosage of a glucocorticoid receptor agonist is greater than 100 mg prednisone equivalent per day. Pulse therapy can include greater than or equal to 250 mg prednisone equivalent per day. Methods of the present invention reduce the dosage of a glucocorticoid receptor agonist needed to achieve the beneficial effects of a low, medium, high, very high or pulse dosage of a glucocorticoid receptor agonist.

Suitable dosages ranges of each of a glucocorticoid receptor agonist and/or a PPAR agonist such as a PPARα agonist, a PPARγ agonist, a PPARδ agonist, a dual PPARα/γ agonist and/or a pan PPAR agonist, depending on various factors such as the age of the subject, the severity and type of condition being treated in the subject, the general condition of the subject, the route and form of administration of the composition being administered and the particular composition administered. One of ordinary skill in the art will be able to ascertain a therapeutically effective amount without undue experimentation in view of the present disclosure and what is known in the art.

Administration of a glucocorticoid receptor agonist and/or at least one PPAR agonist according to a method of the present invention includes administration according to a dosage regimen to produce a desired response. For example, one or more dosage units of a glucocorticoid receptor agonist and/or at least one PPAR agonist is administered to a subject at one time in particular embodiments. A suitable schedule for administration of doses depends on several factors including age, weight, gender, medical history and health status of the subject, type of composition used and route of administration, for example. One of skill in the art is able to readily determine a dose and schedule of administration for a particular subject.

Embodiments of the present invention optionally include administration of a pharmacologically active agent in addition to a glucocorticoid receptor agonist and at least one PPAR agonist.

Non-limiting examples of pharmacologically active agents that can be administered according to embodiments of methods of the present invention include non-steroidal anti-inflammatory agents, antibiotics, antivirals, antineoplastic agents, analgesics, antipyretics, antidepressants, antipsychotics, anticancer agents, antihistamines, anti-osteoporosis agents, anti-osteonecrosis agents, antiinflammatory agents, anxiolytics, chemotherapeutic agents, diuretics, growth factors, hormones and vasoactive agents.

Compositions are provided according to embodiments of the present invention which include a glucocorticoid receptor agonist and at least one PPAR agonist as active agents. Optionally, a pharmaceutically acceptable carrier is included. In preferred compositions, a glucocorticoid receptor agonist and at least one PPARα agonist, PPARγ agonist, PPARδ agonist, dual PPARα/γ agonist and/or pan PPAR agonist are each present in an amount which, in combination, is a therapeutically effective amount for treating a glucocorticoid-responsive condition in a subject. In particular embodiments, a composition of the present invention includes a glucocorticoid receptor agonist and at least one PPARα agonist, PPARγ agonist, PPARδ agonist, dual PPARα/γ agonist and/or pan PPAR agonist each present in an amount which, in combination, is 0.1-99.9% of the composition, such as 0.5-95% of the composition, and such as 1-90% of the composition.

In particular embodiments of inventive compositions, the amount of the glucocorticoid receptor agonist is less than an amount of the glucocorticoid receptor agonist necessary to achieve a therapeutic effect if administered without the at least one PPARα agonist, PPARγ agonist, PPARδ agonist, dual PPARα/γ agonist and/or pan PPAR agonist. Thus, in particular embodiments of compositions of the present invention, the amount of the glucocorticoid receptor agonist in a unit dose of the composition is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90%, less than an amount of the glucocorticoid receptor agonist necessary to achieve a therapeutic effect if administered without the at least one PPARα agonist, PPARγ agonist, PPARδ agonist, dual PPARα/γ agonist and/or pan PPAR agonist. The amount of the glucocorticoid receptor agonist in a unit dose of the composition can be less than 5% or more than 90%, less than an amount of the glucocorticoid receptor agonist necessary to achieve a therapeutic effect if administered without the PPARα agonist, PPARγ agonist, PPARδ agonist, dual PPARα/γ agonist and/or pan PPAR agonist.

In particular embodiments of inventive compositions, the amount of the glucocorticoid receptor agonist is less than an amount of the glucocorticoid receptor agonist necessary to achieve a therapeutic effect if administered without the at least one PPARα agonist. Thus, in particular embodiments of compositions of the present invention, the amount of the glucocorticoid receptor agonist in a unit dose of the composition is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90%, less than an amount of the glucocorticoid receptor agonist necessary to achieve a therapeutic effect if administered without the at least one PPARα agonist. The amount of the glucocorticoid receptor agonist in a unit dose of the composition can be less than 5% or more than 90%, less than an amount of the glucocorticoid receptor agonist necessary to achieve a therapeutic effect if administered without the at least one PPARα agonist.

The amount of a PPARα agonist, PPARγ agonist, PPARδ agonist, dual PPARα/γ agonist and/or pan PPAR agonist in a unit dose according to embodiments of compositions of the present invention is sufficient to achieve a desired therapeutic effect.

Compositions according to embodiments of the present invention include, in combination with a glucocorticoid receptor agonist an amount of at least one PPARα agonist, PPARγ agonist, PPARδ agonist, dual PPARα/γ agonist and/or pan PPAR agonist sufficient to reduce a side-effect of administration of a glucocorticoid receptor agonist.

Compositions according to embodiments of the present invention are made by contacting a glucocorticoid receptor agonist and at least one PPARα, PPARγ agonist, PPARδ agonist, dual PPARα/γ agonist and/or pan PPAR agonist. A pharmaceutically acceptable carrier is optionally also brought into contact with the glucocorticoid receptor agonist and PPAR agonist.

Embodiments of compositions of the present invention optionally include one or more pharmacologically active agents in addition to a glucocorticoid receptor agonist and at least one PPAR agonist. A particular combination of a glucocorticoid receptor agonist, at least one PPAR agonist and one or more additional pharmacologically active agents is selected on the basis of various factors, particularly the disease or condition to be treated, the severity of the disease or condition, and the general state of the subject to be treated.

Non-limiting examples of pharmacologically active agents that can be included in compositions of the present invention include non-steroidal anti-inflammatory agents, antibiotics, antivirals, antineoplastic agents, analgesics, antipyretics, antidepressants, antipsychotics, anticancer agents, antidiabetic agents, anti-osteoporosis agents, anti-osteonecrosis agents, antihistamines, antiinflammatory agents, anxiolytics, chemotherapeutic agents, diuretics, growth factors, hormones and vasoactive agents.

In general, methods of the present invention include administration of one or more active agents as pharmaceutical formulations, including those suitable for oral, rectal, nasal, pulmonary, epidural, ocular, otic, intraarterial, intracardiac, intracerebroventricular, intradermal, intravenous, intramuscular, intraperitoneal, intraosseous, intrathecal, intravesical, subcutaneous, topical, transdermal, and transmucosal, such as by sublingual, buccal, vaginal, and inhalational, routes of administration.

A pharmaceutical composition of the present invention may be in any dosage form suitable for administration to a subject, illustratively including solid, semi-solid and liquid dosage forms such as tablets, capsules, powders, granules, suppositories, pills, solutions, suspensions, ointments, lotions, creams, gels, pastes, sprays and aerosols. Liposomes and emulsions are well-known types of pharmaceutical formulations that can be used to deliver an pharmaceutical agent, particularly a hydrophobic pharmaceutical agent. Pharmaceutical compositions of the present invention generally include a pharmaceutically acceptable carrier such as an excipient, diluent and/or vehicle. Delayed release formulations of compositions and delayed release systems, such as semipermeable matrices of solid hydrophobic polymers can be used.

Pharmaceutically acceptable carriers, methods for making pharmaceutical compositions and various dosage forms, as well as modes of administration are well-known in the art, for example as detailed in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

A pharmaceutical formulation of a composition of the present invention can include a pharmaceutically acceptable carrier. The term “pharmaceutically acceptable carrier” refers to a carrier which is suitable for use in a subject without undue toxicity or irritation to the subject and which is compatible with other ingredients included in a pharmaceutical composition.

A solid dosage form for administration or for suspension in a liquid prior to administration illustratively includes capsules, tablets, powders, and granules. In such solid dosage forms, one or more active agents, is admixed with at least one carrier illustratively including a buffer such as, for example, sodium citrate or an alkali metal phosphate illustratively including sodium phosphates, potassium phosphates and calcium phosphates; a filler such as, for example, starch, lactose, sucrose, glucose, mannitol, and silicic acid; a binder such as, for example, carboxymethylcellulose, alignates, gelatin, polyvinylpyrrolidone, sucrose, and acacia; a humectant such as, for example, glycerol; a disintegrating agent such as, for example, agar-agar, calcium carbonate, plant starches such as potato or tapioca starch, alginic acid, certain complex silicates, and sodium carbonate; a solution retarder such as, for example, paraffin; an absorption accelerator such as, for example, a quaternary ammonium compound; a wetting agent such as, for example, cetyl alcohol, glycerol monostearate, and a glycol; an adsorbent such as, for example, kaolin and bentonite; a lubricant such as, for example, talc, calcium stearate, magnesium stearate, a solid polyethylene glycol or sodium lauryl sulfate; a preservative such as an antibacterial agent and an antifungal agent, including for example, sorbic acid, gentamycin and phenol; and a stabilizer such as, for example, sucrose, EDTA, EGTA, and an antioxidant.

Solid dosage forms optionally include a coating such as an enteric coating. The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied having a thickness such that the entire coating does not dissolve in the gastrointestinal fluids at pH below 3 associated with stomach acids, yet dissolves above pH 3 in the small intestine environment. It is expected that any anionic polymer exhibiting a pH-dependent solubility profile is readily used as an enteric coating in the practice of the present invention to achieve delivery of the active agent to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particular enteric coating material includes acrylic acid polymers and copolymers described for example U.S. Pat. No. 6,136,345.

The enteric coating optionally contains a plasticizer to prevent the formation of pores and cracks that allow the penetration of the gastric fluids into the solid dosage form. Suitable plasticizers illustratively include, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g. hydroxypropylcellulose, acids or bases) to solubilize or disperse the coating material, and to improve coating performance and the coated product.

Liquid dosage forms for oral administration include one or more active agents and a pharmaceutically acceptable carrier formulated as an emulsion, solution, suspension, syrup, or elixir. A liquid dosage form of a composition of the present invention may include a colorant, a stabilizer, a wetting agent, an emulsifying agent, a suspending agent, a sweetener, a flavoring, or a perfuming agent.

For example, a composition for parenteral administration may be formulated as an injectable liquid. Examples of suitable aqueous and nonaqueous carriers include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of a desirable particle size in the case of dispersions, and/or by the use of a surfactant, such as sodium lauryl sulfate. A stabilizer is optionally included such as, for example, sucrose, EDTA, EGTA, and an antioxidant.

For topical administration, a composition can be formulated for administration to the skin such as for local effect, and/or as a “patch” formulation for transdermal delivery. Pharmaceutical formulations suitable for topical administration include, for example, ointments, lotions, creams, gels, pastes, sprays and powders. Ointments, lotions, creams, gels and pastes can include, in addition to one or more active agents, a base such as an absorption base, water-removable base, water-soluble base or oleaginous base and excipients such as a thickening agent, a gelling agent, a colorant, a stabilizer, an emulsifying agent, a suspending agent, a sweetener, a flavoring, or a perfuming agent.

Transdermal formulations can include percutaneous absorption enhancers such as acetone, azone, dimethyl acetamide, dimethyl formamide, dimethyl sulfoxide, ethanol, oleic acid, polyethylene glycol, propylene glycol and sodium lauryl sulfate. Ionotophoresis and/or sonophoresis can be used to enhance transdermal delivery.

Powders and sprays for topical administration of one or more active agents can include excipients such as talc, lactose and one or more silicic acids. Sprays can include a pharmaceutical propellant such as a fluorinated hydrocarbon propellant, carbon dioxide, or a suitable gas. Alternatively, a spray can be delivered from a pump-style spray device which does not require a propellant. A spray device delivers a metered dose of a composition contained therein, for example, using a valve for regulation of a delivered amount.

Opthalmic formulations of one or more active agents can include ingredients such as a preservative, a buffer and a thickening agent.

Suitable surface-active agents useful as a pharmaceutically acceptable carrier or excipient in the pharmaceutical compositions of the present invention include non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, non-substituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, non-substituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of allylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidylcholine, dipalmitoylphoshatidyl-choline and their mixtures.

Suitable non-ionic surfactants useful as pharmaceutically acceptable carriers or excipients in the pharmaceutical compositions of the present invention include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants useful as pharmaceutically acceptable carriers or excipients in the pharmaceutical compositions of the present invention include quaternary ammonium salts, preferably halides, having 4 hydrocarbon radicals optionally substituted with halo, phenyl, substituted phenyl or hydroxy; for instance quaternary ammonium salts containing as N-substituent at least one C8-C22 alkyl radical (e.g. cetyl, lauryl, palmityl, myristyl, oleyl and the like) and, as further sub-stituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2nd ed. (Hanser Verlag, Vienna, 1981) and “Encyclopaedia of Surfactants (Chemical Publishing Co., New York, 1981).

Structure-forming, thickening or gel-forming agents may be included into the pharmaceutical compositions and combined preparations of the invention. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

Detailed information concerning customary ingredients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

Kits according to embodiments of the present invention include a glucocorticoid receptor agonist and one or more PPAR agonists. Kits can include a composition including both a glucocorticoid receptor agonist and at least one PPAR agonist. Instructions for administering a glucocorticoid receptor agonist and the at least one PPAR agonist for treatment of a glucocorticoid-responsive condition in a subject are included in preferred embodiments of an inventive kit.

Embodiments of inventive compositions and methods are illustrated in the following examples. These examples are provided for illustrative purposes and are not considered limitations on the scope of inventive compositions and methods.

### EXAMPLES

### Example 1

### Reagents

DEX, fenofibrate (FF, also abbreviated FENO herein) and WY are all obtained from Sigma-Aldrich. GW647 and GW9578 are previously described (17). Anti-GR, anti-PPARα, anti-RNA pol II and anti-PARP Abs are from Santa Cruz Biotechnology, Inc., Santa Cruz, Calif.

PPARα agonists. WY-14643 (WY), EC₅₀ for human PPARα: 5 μM, for mouse PPARα: 0.63 μM; GW9578, EC₅₀ for human PPARα: 50 nM, for mouse PPARα: 5 nM; GW647, EC₅₀ for human PPARα: 6 nM, for mouse PPARα: 5 nM; and fenofibrate, EC₅₀ for human PPARα: 30 μM, for mouse PPARα: 18 μM.

### Example 2

### Plasmids

p(GRE)₂-50-luc (also called p(GRE)₂50hu.IL6P-luc)) is cloned by replacing the NFkappaB motifs in p(IL6kappaB)₃50hu.IL6P-luc with two consensus GRE sites via PstI-BglII (6). The synthetic reporter construct p(IL6kappaB)₃50hu.IL6P-luc is obtained by replacing the PstI-SspI promoter fragment by a 5′-PstI-blunt-3′ synthetic double-stranded DNA, leaving the proximal 50 bp of the IL-6 promoter. p(IL6kappaB)₃50hu.IL6P-luc refers to a concatenated trimer of the wild-type sequence atgtGGGATTTTCCcatg. pSG5 mPPARα is previously described ((12) and Isseman, I., Prince, R., Tugwood, J. & Green, S., 1992, Biochem Soc. Trans., 20(4):824-827)). pSVhGRα, the expression plasmid for human GRα and pMMTV-Luc, a reporter gene containing the glucocorticoid-responsive mouse mammary tumour virus promoter, are generous gifts from Dr. F. Claessens (KUL, Leuven, Belgium).

### Example 3

### Cell Culture

L929sA and HEK293T cells are maintained in DMEM plus 5% NCS, 5% FCS, 100 U/ml penicillin and 0.1 mg/ml streptomycin. BWTG3 and A549 cells are grown in DMEM plus 10% FCS, 100 U/ml penicillin and 0.1 mg/ml streptomycin. Human hepatoma HepG2 cells are cultured likewise plus 1% non-essential amino acids. Rat FTO2B hepatoma cells are maintained in DMEM:F-12 (1:1) (Invitrogen) plus 10% FCS, 100 U/ml penicillin and 0.1 mg/ml streptomycin. All cell lines are verified to endogenously express GRα and PPARα receptors.

### Example 4

### Isolation of Primary Mouse Hepatocytes

Mouse hepatocytes are isolated by collagenase perfusion from livers of wild type and PPARα KO (PPARα−/−) mice essentially using the collagenase method (18), with several modifications. Mouse livers are perfused with Hanks' balanced salt solution (HBSS, Sigma) at a rate of 5 ml/min via cave vein before addition of collagenase Type IV (0.025%, Sigma). Cell viability is assessed by a Trypan Blue exclusion test. Hepatocytes are cultured as a monolayer on collagen-coated plates in Willam's E medium (Invitrogen) supplemented with 2 mmol/l glutamine, 25 μg/ml gentamycine, 50 nmol/l dexamethasone, 0.1% fatty acid-free bovin serum albumine (BSA; Sigma, France) and 2% ULTROSER (Biosepra, Pall, France) at 37° C. in a humidified atmosphere of 5% CO2. After 2 h, cells are incubated with fresh William's E medium described above without ULTROSER and dexamethasone. After overnight incubation, cells are incubated in a fresh William's E medium supplemented with different compounds, DEX and/or PPARa agonists.

### Example 5

### Transfection Assays

HepG2 and BWTG3 cells are transiently transfected using Lipofectamine according to the manufacturers instructions, HEK293T cells using CaPO₄. At day −1 40,000-50,000 cells/24-well are seeded. At day 0, medium is replaced by 360 μl of fresh normal medium with 10% serum to the cells. The DNA mix is prepared by dissolving (per 24-well) 400 ng of DNA in 20 μl of TE/CaCl2 solution. The DNA-containing mixture is added dropwise to 20 μl BS/Hepes mixture. All is mixed until a fine precipitate is visible. This precipitate is finally added onto the 360 μl medium. After 8 h, medium is replaced with fresh normal medium with 10% serum and inductions are performed the following day. Stable transfection of L929sA cells is performed by the CaPO₄ procedure (19), using a 10-fold excess of the plasmid of interest over the selection plasmid pPGKGeobpA. Transfected cells are selected in 500 μg/ml G418 for 2 weeks, after which the resistant cell clones are pooled for further experiments. In this way, the individual clonal variation in expression is averaged, thus providing a reliable response upon induction. The cotransfected plasmid pPGKGeobpA, conferring resistance to G418 and expressing constitutive β-galactosidase enzymatic activity, is further used as an internal control for calculating the protein concentration.

### Example 6

### Reporter Gene Analysis

Luc and β-gal assays are carried out according to instructions of the manufacturer (Promega). Luc activity, expressed in arbitrary light units, is corrected for the protein conc. in the sample by normalization to constitutive β-gal levels. β-gal levels are quantified with a chemiluminescent reporter assay Galacto-Light kit (TROPIX, Bedford, Mass.).

### Example 7

### RNA Analysis

RNA extraction is performed as described before (12). RNA is isolated from cells by using TRIzol reagent (Invitrogen) according to the manufacturer's instructions. The reverse transcriptase reaction is done by using MLV enzyme (Promega) followed by a PCR reaction with Taq polymerase (Promega) on the obtained cDNA. cDNA is analysed either by a semi-quantitative PCR using Taq polymerase (Promega) or by real-time PCR with a SYBR Green mastermix (Invitrogen). Primers for QPCR of mIL-6: fwd GAGGATACCACTCCCAACAGACC (SEQ ID No. 1) and rev AAGTGCATCATCGTTGTTCATACA (SEQ ID No. 2); for mGILZ: fwd CCAGTGTGCTCCAGAAAGTGTAAG (SEQ ID No. 31) and rev AGAAGGCTCATTTGGCTCAATCTC (SEQ ID No. 4); for hGILZ: fwd GCGTGAGAACACCCTGTTGA (SEQ ID No. 5) and rev TCAGACAGGACTGGAACTTCTCC (SEQ ID No. 6); for mG6Pase: fwd TGCCAGCCTCATGTATTGGA (SEQ ID No. 7) and rev TTCCTGGTCCATCAACCTGG (SEQ ID No. 8); for rMCP-1: fwd GCCAACTCTCACTGAAGCC (SEQ ID No. 9) and rev GCTGGTGAATGAGTAGCAGC (SEQ ID No. 10); for mMMP-9: TGCCCATTTCGACGACGAC (SEQ ID No. 11) and rev GTGCAGGCCGAATAGGAGC (SEQ ID No. 12). Primers for semi-QPCR of hPLAP: fwd GGCTGCAAGGACATCG (SEQ ID No. 13) and rev CAGTTCAGTGCGGTTCC (SEQ ID No. 14).

### Example 8

### Chromatin Immunoprecipitation (ChIP) Assay

ChIP assays are performed as previously described (12), ChIP assays against GR and polymerase II are performed according to the ChIP kit instructions (Upstate Biotechnology, Lake Placid, N.Y.). Cells are starved for 48 h in serum-free medium, then solvent-treated or treated as described in the figure legends Primers within the GILZ promoter region are from Wang and coworkers (22). Ct-values obtained in the QPCR assays are analysed using GENEX software (BioRad). The relative amount of the precipitated target sequence is determined via normalization to the “input”, i.e. the purified total gDNA levels.

### Example 9

### ELISA

Murine IL-6 ELISA is performed using a kit from Biosource.

### Example 10

### Mice Handling

Female C57BL6J mice are used at 8 weeks. Mice are randomized to four groups (six mice/group) and matched for body weight. Animals are killed by cervical dislocation after which thymus and liver are recovered and weighed. Total RNA is extracted from liver as described below. ANOVA is used for all analyses, followed by Scheffe post-hoc tests for treated vs control comparisons. The level of significance for all statistical analyses is set at p<0.05.

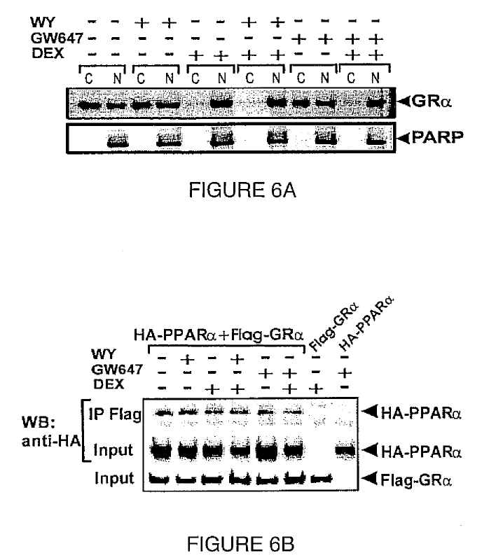

Male C57B16 mice are subject to a high fat diet, containing 36.4% lard (UAR, Epinay, France) for 7 weeks, after which they are randomized to four groups according to weight and blood glucose, and upon which daily treatment with reference compounds as stated in the legend of FIG. 5 is started. After 7 days of treatment, mice are fasted for 6 h, after which an intraperitoneal glucose tolerance test (IPGTT) is performed. Blood Glc levels are determined before and 15, 30, 45, 60 and 90 minutes after Glc injection. Statistical differences are explored via the Mann-Whitney U-test.

### Example 11

### Cytosolic & Nuclear Fractionation, Immunoprecipitation and Western Blotting

Nuclear extracts are prepared as described previously (20). Nuclear lysates are prepared from control and treated cells. Briefly, confluent cells from 10-cm-diameter dishes are washed twice with phosphate-buffered saline. After washing, 5 ml of ice-cold hypotonic lysis buffer is added (20 mM HEPES [pH 7.6], 20% glycerol, 10 mM NaCl, 1.5 mM MgCl2, 0.2 nM EDTA, 0.1% Triton X-100, 25 mM NaF, 25 mM-glycerophosphate, 1 mM phenylmethylsulfonyl fluoride, 1 mM sodium orthovanadate, 1 mM dithiothreitol, and protease inhibitors). The cells are allowed to swell on ice for 5 min before they are scraped and collected. Nuclei are pelleted by centrifugation at 500 rpm in a Beckman swinging-bucket tabletop centrifuge for 5 min and resuspended in 100 to 200 μl of nuclear extraction buffer (hypotonic buffer plus 500 mM NaCl). After incubation and rocking at 4° C., the lysates are cleared of debris by centrifugation. Equal amounts of nuclear and cytoplasmic protein extracts are fractionated by standard SDS-PAGE followed by standard Western Analysis. Nuclear extracts from transfected HEK293T cells are subject to a co-immunoprecipitation protocol adjusted from Adcock et al. (21). 100 μg of protein is incubated with 20 μl of M2 Flag beads (pre-washed 4× with buffer A [10 mM Hepes pH 7.5, 1.5 mM MgCl2, 10 mM KCl, 0.5 mM DTT, 0.1% NP-40 and freshly added protease inhibitors pefabloc and aprotinine] in the presence of 0.5% BSA) and extra buffer A is added to total volume of 300 μl. Rotation at 4° C. (spinning wheel) is done for 18 h (evening to next morning). Beads are washed 4× in buffer A, supplemented with 150 mM NaCl and 0.5% TX-100. 25 μl of 2× Laemmli buffer is added onto beads and the sample is boiled for 1 min at 95° C. Samples are loaded onto a 8% SDS-PAGE gel, together with the inputs of the total cell lysate

### Example 12

### Statistical Analysis

Statistical significance is determined using one way ANOVA tests followed by Dunnett's Multiple Comparison Test. Values of P<0.05 are considered significant.

### Example 13

### PPARα and GRα Cooperate to Inhibit NF-κB-Driven Gene Expression

PPARα and GRα inhibit inflammation through interfering with the activity of NF-κB. Specific PPARα agonists, WY-14643 (WY) and GW647, and the GRα agonist dexamethasone (DEX) are administered to cells separately and together to determine the effects on TNF-induced IL-6 production.

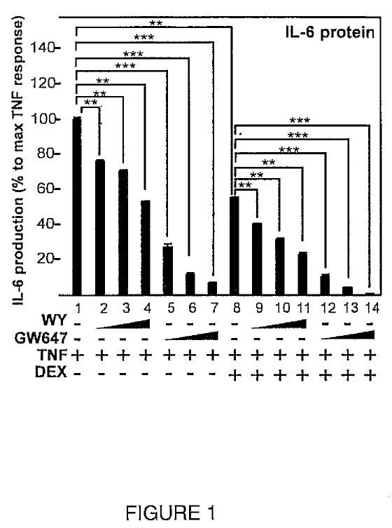

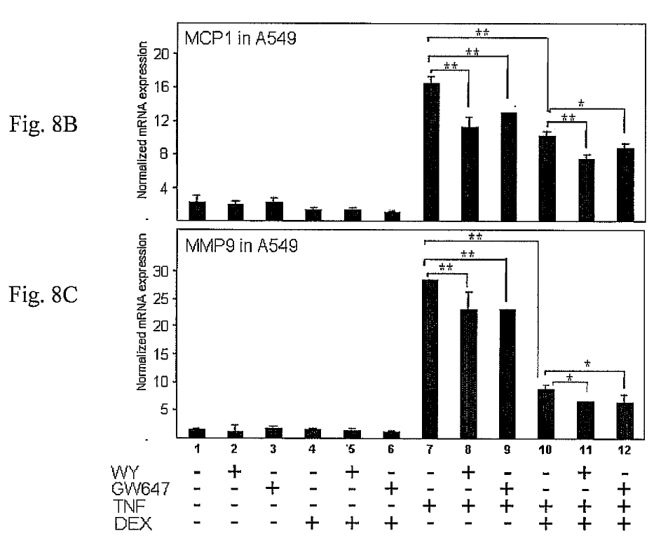

L929sA cells characterized by stably integrated p(IL6 KB)₃50hu.IL6P-luc+ are pre-incubated with solvent, DEX (0.01 μM), GW647 (1, 0.5 or 0.25 μM), WY (2, 5 or 10 μM) or various combinations thereof, for 1 h, before Tumor Necrosis Factor (TNF) (200 IU/ml) is added, where indicated, for 24 h. Medium is collected to perform a murine IL-6 ELISA. Protein levels obtained in ng/ml are calculated as % of max TNF response. Results are shown ±SD. **P<0.01, ***P<0.001 in FIG. 1. Bar 1: indicates results of 200IU/ml TNF application only; bar 2: 2 μM WY+200 IU/ml TNF; bar 3: 5 μM WY+200 IU/ml TNF; bar 4: 10 μM WY+200 IU/ml TNF; bar 5: 0.25 μM GW647+200 IU/ml TNF; bar 6: 0.5 μM GW647+200 IU/ml TNF; bar 7: 1 μM GW647+200 IU/ml TNF; bar 8: 0.01 μM DEX+200 IU/ml TNF; bar 9: 2 μM WY+0.01 μM DEX+200 IU/ml TNF; bar 10: 5 μM WY+0.01 μM DEX+200 IU/ml TNF; bar 11: 10 μM WY+0.01 μM DEX+200 IU/ml TNF; bar 12: 0.25 μM GW647+0.01 μM DEX+200 IU/ml TNF; bar 13: 0.5 μM GW647+0.01 μM DEX+200 IU/ml TNF; and bar 14: 1 μM GW647+0.01 μM DEX+200 IU/ml TNF. The luc assay results are shown in FIG. 8A.

Cells incubated with WY-14643 (WY), GW647 or DEX, separately, display inhibited TNF-induced IL-6 production in a dose-responsive manner. Results of this assay in L929sA cells are shown in FIG. 1. Control experiments with solvent and compounds alone have negligible effects on basal IL-6 production. Administration of both a PPARα agonist and a GRα agonist together activates both PPARα and GRα and results in an additive repression of IL-6 production in L929sA cells. Similar data are obtained using A549 human lung epithelial cells.

L929sA cells, stably transfected with p(IL6 κB)₃50hu.IL6P-luc+, an NF-κB-dependent recombinant promoter construct are treated with WY, GW647, DEX, a combination of WY and DEX or a combination of GW647 and DEX to determine the effects of PPARα and GRα activation on NF-κB-mediated transcription. The results illustrate that NF-κB-mediated transcription is additively inhibited by GCs and PPARα agonists, FIG. 8A. These data are confirmed in A549 cells at the mRNA level, via quantitative RT-PCR (QPCR) analysis, for other inflammatory markers, namely MCP-1 and MMP9, FIGS. 8B and 8C, respectively.

Microarray analysis of RNA isolated from primary murine hepatocytes treated with solvent (control), DEX, GW9578 or DEX and GW957S, demonstrates cooperativity on gene expression regulation of several inflammatory markers, including Ccl2 (MCP-1), Ccl20, Cxc12, Cxc13 and VCAM1, indicating a cell-type independent effect of combined GC and PPARα agonist treatment.

### Example 14

### PPARα Agonists Block Induction of GC-Responsive Genes by Suppression of GRE-Driven Gene Transcription

The effect of different PPARα agonists on GC-induced mRNA expression of GC-inducible genes is measured using semi-quantitative PCR (semi-QPCR) and quantitative PCR (QPCR). The GC-inducible genes contain in their promoter region one or more functional GRE elements onto which GRα binds as a homodimer.

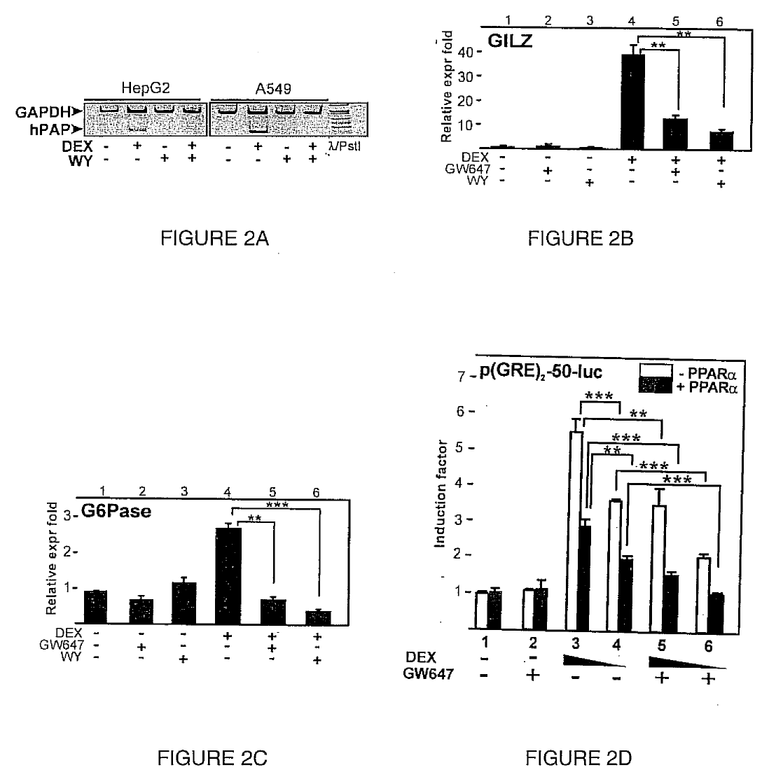

Cells are treated with solvent, DEX (1 μM), GW9578 (500 nM) or WY (10 μM) or various combinations. A549 or HepG2 cells are treated for eight hours, mRNA is isolated, reverse transcribed and the resulting cDNA is subjected to semi-quantitative PCR analysis with primers to detect GAPDH (loading control) or hPAP in the same sample. Results of this assay are shown in FIG. 2A, indicating that DEX upregulates mRNA expression levels of human placental alkaline phosphatase (hPAP) in HepG2 human hepatocyte cells and A549 cells. Treatment with WY alone has no effect on hPAP mRNA expression. Surprisingly, when cells are co-treated with DEX and WY, hPAP mRNA levels are significantly inhibited, as compared to DEX alone, a result shown in FIG. 2A.

Similar results are obtained for other glucocorticoid-inducible genes. HepG2 cells and FTO2B cells are incubated with the indicated agents for three hours. mRNA is isolated, reverse transcribed and the resulting cDNA is subjected to SYBR green QPCR with primers to detect G6Pase or Glucocorticoid-induced Leucine Zipper (GILZ). QPCR measurements are performed in triplicate. QPCR results, normalized to expression of household genes, are shown ±SD, in FIGS. 2B and 2C. Results are represented as relative expression fold, i.e. with the solvent-treated control value taken as 1.

DEX upregulates mRNA expression levels of GILZ in HepG2 human hepatocyte cells and A549 cells. Treatment with WY alone has no effect on GILZ mRNA expression. Surprisingly, when cells are co-treated with DEX and WY, GILZ mRNA levels are significantly inhibited, as compared to DEX alone Glucocorticoid-induced Leucine Zipper (GILZ) in HepG2 cells, as shown in FIG. 2B. Similar results are obtained in A549 cells.

Similar results are also obtained for the glucocorticoid-inducible gene Serum and Glucocorticoid-inducible Kinase 1 (SGK1) in both HepG2 cells and A549 cells using WY or GW647 as PPARα agonists. Further, the combined effect of DEX and the PPARα agonists WY or GW647 results in a significant gene-inhibitory effect on Glucose-6-Phosphatase (G6Pase), a hepatic GC-regulated gene, in FTO2B rat hepatocytes as shown in FIG. 2C. The effects of combined administration of a PPARα agonist and a GRα agonist are thus cell-type and species-independent.

The effect of PPARα ligands on GRα-induced gene expression occurs via interference with GRE-mediated gene transcription as shown by the effect of GW647 on the activity of DEX-induced p(GRE)₂-50-Luc, a recombinant GRE-driven reporter gene DEX, in contrast to GW647, strongly activates the promoter in a dose-dependent manner, shown in FIG. 2D, white bars. However, when combined with GW647, shown in FIG. 2D, black bars, the induction is inhibited, confirming the results of mRNA analysis, exemplified in FIGS. 2A-C.

HepG2 cells are transiently transfected with p(GRE)₂-50-luc, and pSG5PPARα (black bars) or pSG5 (white bars). Twenty-four hours later, cells are treated with solvent, DEX (1 or 0.1 μM, GW647 (500 nM), or various combinations of these agents and concentrations, such as 0.1 μM DEX+500 nM GW647 or 1 μM DEX+500 nM GW647, for a total period of 8 h. Cell lysates are assayed for luciferase (luc) activities and normalized for β-gal activities. Promoter activities are expressed as relative induction factor, i.e., the ratio of expression levels of induced versus non-induced conditions.

Furthermore, overexpression of PPARα, FIG. 2D, black bars, results in a ligand-independent decrease of DEX-induced luciferase (luc) activity. This partially ligand-independent effect is a typical characteristic of PPARα in overexpression systems. The transcriptional inhibition is further enhanced in the presence of GW647, FIG. 2D, black bars. These findings are confirmed using WY and using the MMTV promoter, which contains multiple GREs, stably integrated in L929sA cells. PPARα agonists did not block GRα-mediated gene expression by influencing the level of GRα protein since GRα protein levels, assayed from the same lysates used for the luc measurements, remain unaffected under the various treatment combinations.

### Example 15

### PPARα Agonists Inhibit GC-Induced Gene Expression in Primary Hepatocytes in a PPARα-Dependent Manner

Murine primary hepatocytes isolated from wild type (WT) and PPARα knockout (KO) mice (Gonzalez F J. Recent update on the PPAR alpha-null mouse. Biochimie. 1997 February-March; 79 (2-3):139-144), are used to illustrate that activated PPARα interferes with GR-mediated gene expression.

Primary hepatocytes isolated from PPARα knockout mice or from wild type mice are treated with solvent or GW9578 (500 nM) or WY (10 μM) for 24 h. mRNA is isolated, reverse transcribed and subjected to QPCR with primers to detect PDK-4.

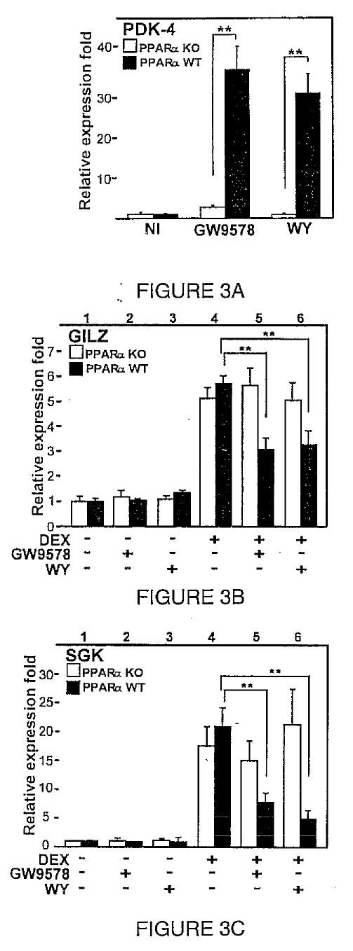

As a positive control, the effect of PPARα ligands is tested on Pyruvate Dehydrogenase Kinase-4 (PDK-4), a representative PPARα target gene. Treatment with GW9578 and WY results in a significant increase in PDK-4 mRNA levels only in WT cells, a result shown in FIG. 3A.

Similar results are obtained for Acyl coA Oxidase (ACO) another Peroxisome Proliferator Response Element (PPRE)-driven target gene.

Primary hepatocytes from PPARα knockout mice or from wild type mice are treated with solvent, GW9578 (500 nM), WY (10 μM), DEX (1 μM) or various combinations thereof, as indicated, for 24 h. mRNA is isolated, reverse transcribed and subjected to QPCR using primers to detect GILZ or SGK1. QPCR measurements are performed in triplicate and the normalized results are represented as expression folds, i.e. taking the control value as 1 and shown ±SD.

GILZ and SGK1 mRNA expression levels are substantially upregulated upon treatment with DEX in primary hepatocytes from both PPARα WT and mutant mice, shown in FIG. 3B, 3C. In WT cells, this induction is significantly inhibited by co-treatment with DEX and WY or DEX and GW9578. In contrast, the PPARα ligands do not affect the GC-induced expression of GILZ or SGK1 in hepatocytes isolated from PPARα KO mice, shown in FIG. 3B, 3C, indicating that the inhibitory effect of the PPARα ligands is PPARα-dependent.

### Example 16

### PPARα Agonists Inhibit GC-Induced Gene Expression in vivo

The effect of the PPARα agonist fenofibrate (FF) in vivo is determined by assaying the levels of GILZ and ACO mRNA in mouse liver.

Groups of 6 mice per group, randomized according to their weight, are treated with either DEX (10 mg/kg, i.p.) or an equal volume of normal saline, and/or FF (200 mg/kg, gavage) or an equal volume of 0.5% CMC (control) every day for a period of 5 days. GILZ and ACO mRNA expression levels from the liver are quantified via QPCR and normalized for household gene expression. Results from triplicates are shown ±SD. Results are represented as relative expression fold, i.e. with the solvent-treated control value taken as 1.

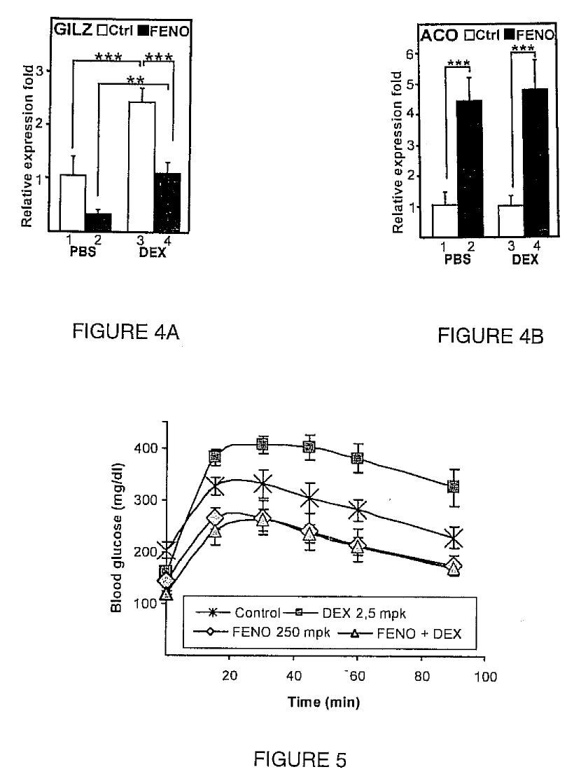

Results of these treatments are shown in FIG. 4A. DEX-treated mice show a significant increase in GILZ mRNA levels compared to the control group (P<0.0001). Unexpectedly, co-treatment with DEX and FF significantly inhibits GILZ mRNA levels as compared to DEX alone (P<0.0001). Similar results are also obtained for SGK1.

A decrease in basal GILZ mRNA gene expression is also apparent in FF-treated mice as compared to control mice, an effect most likely caused by the antagonism of activated PPARα on basal levels of GILZ expression by endogenously and systemically present GCs, in line with an in vivo PPARα and GRα cross-talk.

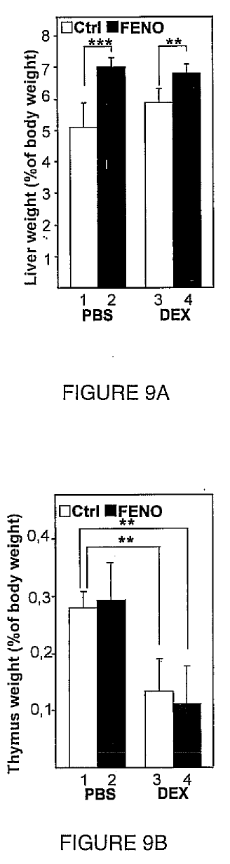

As a positive control for the activity of FF, ACO mRNA expression, FIG. 4B, as well as liver weights, FIG. 9A, are measured. GC-induced loss of thymus weight is unaffected by FF treatment in addition to DEX treatment, FIG. 9B. DEX treatment alone has no effect on ACO mRNA, whilst treatment with FF results in a significant induction of ACO mRNA levels. Simultaneous treatment with both FF and DEX has no additional effect compared to FF alone, FIG. 4B.

### Example 17

### PPARα Antagonizes Both High Fat Diet and GC-Mediated Insulin Resistance in vivo

Antagonism between GRα and PPARα has clinical importance with respect to the development of insulin resistance. The influence of DEX and/or FF on glucose homeostasis is shown in an insulin-resistant high fat diet fed mouse model.

Groups of 6 mice per group with an acquired insulin resistance through the intake of a high fat diet for 7 weeks, are daily treated with either PBS (control), DEX (2.5 mg/kg), FF (200 mg/kg) or DEX/FF combined, for 7 days, after which an intraperitoneal Glc tolerance test is performed, measuring blood Glc levels before and 15, 30, 45, 60 and 90 minutes after a Glc injection.

Results are shown ±SD in FIG. 5, *P<0.05. Treatment with DEX for 7 days aggravates the insulin resistance phenotype, measured by an intraperitoneal glucose tolerance test (IPGTT). Treatment with the PPARα agonist FF improves glucose tolerance. Surprisingly, the combination of DEX with FF completely prevented the DEX-mediated insulin resistance.

Groups of 6 mice per group with an acquired insulin resistance through the intake of a high fat diet for 7 weeks, are daily treated with either PBS (control), DEX (2.5 mg/kg), FF (100 mg/kg) or DEX (2.5 mg/kg)/FF combined, for 7 days, after which an intraperitoneal Glc tolerance test is performed, measuring blood Glc levels before and 15, 30, 45, 60 and 90 minutes after a Glc injection, to obtain similar results.

Groups of 6 mice per group with an acquired insulin resistance through the intake of a high fat diet for 7 weeks, are daily treated with either PBS (control), DEX (2.5 mg/kg), clofibrate (200 mg/kg) or DEX (2.5 mg/kg)/clofibrate combined, for 7 days, after which an intraperitoneal Glc tolerance test is performed, measuring blood Glc levels before and 15, 30, 45, 60 and 90 minutes after a Glc injection, to obtain similar results.

Groups of 6 mice per group with an acquired insulin resistance through the intake of a high fat diet for 7 weeks, are daily treated with either PBS (control), DEX (2.5 mg/kg), gemfibrozil (200 mg/kg) or DEX (2.5 mg/kg)/gemfibrozil combined, for 7 days, after which an intraperitoneal Glc tolerance test is performed, measuring blood Glc levels before and 15, 30, 45, 60 and 90 minutes after a Glc injection, to obtain similar results.

Groups of 6 mice per group with an acquired insulin resistance through the intake of a high fat diet for 7 weeks, are daily treated with either PBS (control), DEX (2.5 mg/kg), gemfibrozil (200 mg/kg), DEX (2.5 mg/kg)/5 μM rosiglitazone or DEX (2.5 mg/kg)/10 μM rosiglitazone combined, for 7 days, after which an intraperitoneal Glc tolerance test is performed, measuring blood Glc levels before and 15, 30, 45, 60 and 90 minutes after a Glc injection, to obtain similar results.

Groups of 6 mice per group with an acquired insulin resistance through the intake of a high fat diet for 7 weeks, are daily treated with either PBS (control), DEX (2.5 mg/kg), gemfibrozil (200 mg/kg) or DEX (2.5 mg/kg)/10 μM CpdA (H. C. Owen, et al., Mol Cell Endocrinol 264 (2007), pp. 164-170) or DEX (2.5 mg/kg)/10⁻⁶M AL-438 (De Bosscher K, et al., Proc Natl Acad Sci USA. 2005 Nov. 1; 102(44):15827-32) combined, for 7 days, after which an intraperitoneal Glc tolerance test is performed, measuring blood Glc levels before and 15, 30, 45, 60 and 90 minutes after a Glc injection, to obtain similar results.

### Example 18

### Activated PPARα and GRα Interact in the Nucleus

GRα moves from the cytoplasm to the nucleus upon hormone binding and present results show that activated PPARα does not influence the subcellular localization of activated GRα.

A cellular fractionation assay in BWTG3 cells treatment with is performed. After serum starvation in phenol red-free medium for 24 h, BWTG3 cells are treated with solvent (NI) or induced with DEX (1 μM), WY (50 μM), GW647 (500 nM) or various combinations thereof for 1 h upon which cells are subjected to a cellular fractionation assay. Western blot analysis is performed using an anti-GR Ab. Simultaneous probing with an anti-PARP Ab serves as a control for the fractionation efficiency. The displayed bands are blotted onto two different membranes.

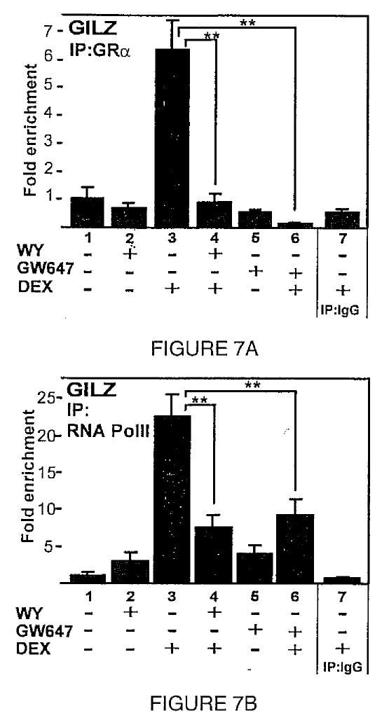

In untreated or PPARα agonist-treated cells, a majority of GRα protein resides in the cytoplasm, although a substantial amount is also present in the nucleus as shown in FIG. 6A; C, cytoplasmic; N, nuclear. DEX stimulation for 1 h leads to a mainly nuclear GRα distribution, which remained unaffected by co-treatment with PPARα ligands. PPARα is found to be predominantly nuclear, regardless of the treatment.

Equal amounts of differently tagged receptor variants are transfected in HEK293T cells. Cells are stimulated with various agents separately and in combination as indicated in FIG. 6B, followed by co-immunoprecipitation analysis of the nuclear fraction using anti-Flag beads and immunoblotting with an anti-HA ab. Input controls for Flag-GRα and HA-PPARα are verified by Western blot analysis using anti-Flag and anti-HA, respectively. A representative of two independent experiments is shown.

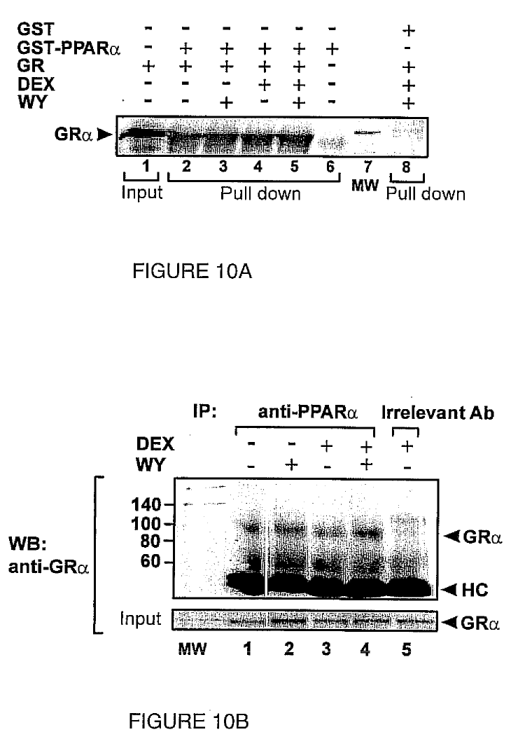

Co-immunoprecipitation analysis using nuclear extracts of HEK293T cells in which differently tagged receptor variants, Fla-GRα and HA-PPARα, are overexpressed, demonstrating that PPARα and GRα can physically interact. Unexpectedly, however, this interaction is ligand-independent. This finding is confirmed in GST-pull down and in immunoprecipitation assays of endogenous proteins using BWTG3 cells, FIGS. 10A and 10B, respectively.

### Example 19

### PPARα Agonists Interfere with the Recruitment of Activated GRα at a Classical GRE-Containing Promoter

ChIP assays are performed using primer pairs encompassing the classical GRE in the GILZ promoter to determine whether activated PPARα interferes with the recruitment of activated GRα on GRE-driven promoters.

Following serum starvation for 48 h, A549 cells are incubated with solvent, DEX (1 μM), WY (50 μM), GW647 (500 nM) or various combinations for 2 h.

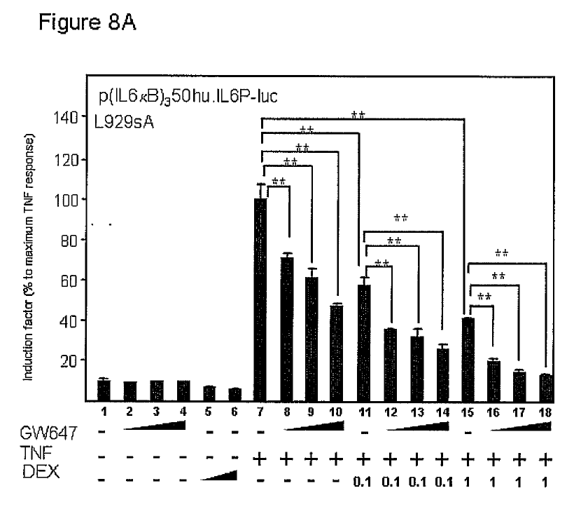

Cross-linked and sonicated cell lysates are subjected to ChIP analysis against GR or RNA polymerase II (RNA pol II). QPCR is used to assay recruitment at the GILZ gene promoter. The quantity of GR or RNA pol II detected on the GILZ promoter is shown in FIGS. 7A and 7B, respectively, with a correction of the SYBR green QPCR signal for input control. Lanes 1-6 are performed with the specific Ab, as indicated in the graph; lane 7 includes the IgG control. The reaction is performed in triplicate.

No GRα occupancy is observed in either solvent-treated or PPARα agonist-treated cells, whereas a significant GRα recruitment is observed upon DEX stimulation, FIG. 7A. In contrast, co-treatment with the PPARα ligands WY or GW647 abrogates DEX-induced GRα recruitment.

RNA pol II recruitment, a marker for induced promoter activity, is also enhanced upon DEX stimulation, whereas combination treatment of DEX and PPARα ligands inhibits this recruitment significantly, FIG. 7B, correlating with the recruitment pattern observed for GRα. The fact that activated PPARα interferes with GRα− and concomitant RNA pol II-promoter recruitment provides a mechanistic basis for the gene-repressive effects of activated PPARα on GRα-mediated gene transcription.

### Example 20

C2C12 muscle cells are treated with solvent, DEX (0.01 μM), GW647 (1, 0.5 or 0.25 μM), WY (2, 5 or 10 μM) or combinations thereof, for 24 h. Combinations include 2 μM WY+0.01 μM DEX; 5 μM WY+0.01 μM DEX; 10 μM WY+0.01 μM DEX; 0.25 μM GW647+0.01 μM DEX; 0.5 μM GW647+0.01 μM DEX; and 1 μM GW647+0.01 μM DEX. mRNA extraction is performed, followed by generation of cDNA and QPCR analysis for muscle markers including: glutamine synthetase, GLUT4, myogenin, PGC1a, and UCP3.

### Example 21

3T3L1 adipocyte cells are treated with solvent, DEX (0.01 82 M), GW647 (1, 0.5 or 0.25 μM), WY (2, 5 or 10 μM or combinations thereof, for 24 h. Combinations include 2 μM WY+0.01 μM DEX; 5 μM WY+0.01 μM DEX; 10 μM WY+0.01 μM DEX; 0.25 μM GW647+0.01 μM DEX; 0.5 μM GW647+0.01 1 μM DEX; and 1 μM GW647+0.01 μM DEX. mRNA extraction is performed, followed by generation of cDNA and QPCR analysis fat cell markers including: adiponectin, aP2, LPL, and adipsin.

### Example 22

In vivo assays are performed to determine reversal of insulin resistance in vivo and to measure the effect of PPARα agonists on other GC-dependent target genes in vivo

C57B16 male mice are used. Mice designated EXP1 are fed a Standard chow diet (E113; UAR, Epinay, France) throughout the treatment. Mice designated EXP2 are first subjected to a high fat diet, containing 36.4% lard (UAR, Epinay, France) for 7 weeks, after which they are randomized to four groups according to weight and blood glucose. PBS (control), DEX (2.5 mg/kg), FF (200 mg/kg) or DEX/FF combined are administered by intraperitoneal injection once a day (50-100 μl of the formulated compound per 20 g of mice) at 9 a.m on one subgroup with fasted and one subgroup with non-fasted mice. The vehicle used is Phosphate Buffer Saline (PBS)

Day-3: the mice are weighed (9 a.m) and blood glucose is determined (by tail nicking in conscious mice). For the fasted mice group, food is removed overnight and blood samples are performed (9 a.m) after about 16 hour-period fasting by sinus retroorbital punction under isoflurane anaesthesia

Parameters in blood: triglycerides, total cholesterol, HDL-cholesterol, free fatty acids, insulinemia and blood glucose determination.

Randomization of the mice happens according to their body weight and blood glucose. EXP1: 8 groups of 6 mice: 1) Standard diet/non-fasted/PBS control2) Standard diet/non-fasted/GCs 3) Standard diet/non-fasted/PPAR agonists 4) Standard diet/non-fasted/GCs+PPAR agonists, 5) Standard diet/fasted/PBS control 6) Standard diet/fasted/GCs 7) Standard diet/fasted/PPAR agonists 8) Standard diet/fasted/GCs+PPAR agonists

EXP2: 8 groups of 6 mice 1) High-fat diet/non-fasted/PBS control 2) High-fat diet /non-fasted/GCs 3) High-fat diet/non-fasted/PPAR agonists 4) High-fat diet/non-fasted/GCs+PPAR agonists, 5) High-fat diet/fasted/PBS control 6) High-fat diet/fasted/GCs 7) High-fat diet/fasted/PPAR agonists 8) High-fat diet/fasted/GCs+PPAR agonists. Throughout the treatment, the mice are weighed twice a week (not fasted)

Day 7 of treatment: intraperitoneal glucose tolerance test (IPGTT) and an insulin-tolerance test (ITT) on mice for glucose determination at 0, 15, 30, 60 and 90 minutes after the glucose injection (blood samples by tail cutting in conscious mice)

This test is performed on either non-fasted mice or mice fasted for about 16 hours before the experiment.

Day 10: the mice are weighed. Blood samples are performed after a 16 hour-period fasting (2 p.m) by sinus retroorbital punction under isoflurane anaesthesia for triglycerides, cholesterol, HDL-cholestero, free fatty acids, insulinemia and blood glucose determination.

The mice are sacrificed by cervical dislocation. Liver, epididymal, peri-renal and inguinal (interscapular), thymus and pancreas are weighed. Muscles are collected. Half of the collected tissues are frozen in liquid nitrogen, the other half is collected in a commercially available tissue storage reagent: RNALATER.

mRNA is isolated from tissues collected, and cDNA is generated. Gene expression regulation is analyzed through QPCR analysis of glucose-6-phosphatase, PEPCK, TAT, FOXO1, sgk, Hsp27, Gpx3, GILZ, alpha-fetoprotein, CPT-1, PDK4, and ACO as well as muscle genes glutamine synthetase, GLUT4, myogenin, PGC1a and UCP3 and adipocyte tissue genes adiponectin, aP2, LPL and adipsin.

The ANOVA is used for all analyses, followed by scheffe post-hoc tests for treated vs control comparisons. The level of significance for all statistical analyses is set at p<0.05.

### Example 23

In vivo, in two distinct murine models of obesity abnormally elevated levels of JNK activity is detected. These elevated levels are inhibited in peripheral tissues by rosiglitazone, a PPARgamma agonist. Moreover, rosiglitazone fails to enhance insulin-induced glucose uptake in primary adipocytes from ob/ob JNK1−/− mice. Accordingly, the hypoglycemic action of rosiglitazone is abrogated in diet-induced obese JNK1-deficient mice. A mechanism based on targeting the JNK signaling pathway, is involved in the hypoglycemic and potentially in the pancreatic beta-cell protective actions of TZDs/PPARgamma Díaz-Delfín J, Morales M, Caelles C., Diabetes. 2007, 56(7):1865-1871). The effects of glucocorticoid agonists and PPARαagonists on JNK kinase relating to the combined hypoglaecemic effect and determination of glucose transport are determined.

Eight week-old male ob/ob, ob/ob JNK1−/−, and lean mice are treated with GCs, PPARα agonists, GCs+PPARα agonists or vehicle, once a day, for 4 consecutive days. Epididymal fat pads are dissected, minced in Krebs-Ringer solution supplemented with 2 mmol/l sodium pyruvate and 3% BSA, and digested with 1.5 mg/ml collagenase. Adipocytes are filtered, washed three times in the same buffer, and placed in plastic vials in a final volume of 400 μl. In triplicates, cells are treated with vehicle, GCs, PPARα agonists, GCs and PPARα agonists, for example: PBS (control), DEX (2.5 mg/kg, FF (200 mg/kg) or DEX/FF combined, in absence or presence of insulin, for 10 min at 37° C. before 2-deoxy-D-[3H]glucose (2-DG) is added at a final concentration of 0.1 mmol/l (0.4 μCi). After 10 min, 100 μl of 100 μmol/l cytochalasin B is added, and adipocytes are separated by centrifugation in microtubes containing phthalic acid dinonyl ester (density 0.98 g/ml). Incorporation of labeled 2-DG is measured by liquid scintillation.

### Example 24

GILZ is one example of a GC-induced gene that may mediate part of the anti-inflammatory effects of GCs, especially in immune cells. SGK1, another gene controlled by GCs via a GRE-element in its 5′-region, is together with GILZ believed to be involved in the regulation of tonic inhibition of α-epithelial Na channels. The involvement of SGK1 in the cell surface redistribution of α-epithelial Na channels further explains why sustained high levels of the protein and its activity may contribute to conditions such as hypertension and diabetic nephropathy. Both proteins are also able to propagate the rapid effects of the mineralocorticoid hormone aldosterone, an effect contributing to increased sodium reabsorption, and on its turn linked to hypertension. Together with the diabetogenic effect of GC excess, the increased expression of these factors may further contribute to an increased cardiovascular risk in patients that are highly dependent on a chronic steroid treatment. The effects of methods and compositions of the present invention on GILZ and SGK1, both proteins involved in processes that regulate sodium reabsorption, support use of PPAR agonists to lower GC-induced hypertension.

Methods and compositions of the present invention are used to treat glucocorticoid-induced hypertension in two mouse models of hypertension, the renovascular two-kidney, one clip model and the mineralocorticoid deoxycorticosterone-salt model, described in detail in Johns, C et al., Hypertension. 1996; 28:1064-1069.

Hypertension, defined as systolic pressures higher than 140 mm Hg, is developed in more than 50% of mice so treated. Indirect tail-cuff blood pressure measurements as well as direct intra-arterial monitoring of blood pressure in conscious, freely moving mice is used to monitor the effects of administered compounds including solvent, DEX (0.01 μM), GW647 (1, 0.5 or 0.25 μM), WY (2, 5 or 10 μM) or combinations thereof, for 24 h. Combinations include 2 μM WY+0.01 μM DEX; 5 μM WY+0.01 μM DEX; 10 μM WY+0.01 μM DEX; 0.25 μM GW647+0.01 μM DEX; 0.5 μM GW647+0.01 μM DEX; and 1 μM GW647+0.01 μM DEX.

### Example 25

Glucocorticoid-induced osteoporosis (GIO) has been considered one of the most debilitating side-effects related to long-term GC usage (Berris, Repp et al. Curr Opin Endocrinol Diabetes Obes 14(6): 446-50). The effects of compositions and methods of the present invention on glucocorticoid-induced osteoporosis is determined by analysis of markers of osteoclastogenesis, including cathepsin K, M-CSF, RANKL and OPG. Since it is believed that an increase in bone resorption is worsened by inhibition of new bone formation, thereby contributing to the GC-mediated decrease in bone mineral density, the effect compositions and methods of the present invention on osteoblast differentiation is determined using calvarial cells isolated from 3-5 day old mice.

Alkaline phosphatase staining and Q-PCR are performed for the detection of Col1a1, Alkp, Runx-2 and Bglap (osteocalcin) expression after 10 days of osteoblast differentiation. Alizarin Red staining is performed to determine extracellular calcium deposition after 20 days of osteoblast differentiation.

Ex vivo

Differentiation of Osteoblasts from Calvarial Cells:

Calvarial cells are isolated from 3-5 day old mice (SV 129 background). A piece of the tail is isolated for genotyping. The pups are decapitated with scissors in the laminar flow cabinet, skin and brain are removed and the calvaria transferred into eppendorf tubes containing 1 ml PBS+1% Pen/Strep. The tubes are put on ice until digestion. For the digestion, the PBS is replaced with 1 ml digestion solution (α-MEM containing 1% Pen/Strep, 0.1% Collagenase A and 0.2% Dispase II, dissolved by agitation and filtered) and shaken for 10 min at 37° C. (<700 rpm). The liquid phase is then removed. The digestion is repeated another 4 times, and fractions 2 until 5 are collected, keeping them on ice. The digested fractions are spun down and one calvaria is plated into one 6-well, containing α-MEM supplemented with 10% FCS, 1% Gln and 1% Pen/Strep. The medium is changed the following day keeping the cells below a confluency of 80%. When cells have reached almost 80% of confluence and genotyping is performed, cells can be pooled and seeded for subsequent experiments.

Induction of Osteoblast Differentiation:

Mineralization medium consists of α-MEM, supplemented with 100 μg/ml ascorbic acid and 5 mM β-glycerolphosphate, whether or not supplemented with one or more glucocorticoid receptor agonist (e.g. DEX) or glucocorticoid receptor agonist +PPAR agonist combinations.

Alkaline Phosphatase (ALP) Staining

The cell medium is discarded and 0.5 ml fixation solution (dilute 1 ml concentrated citrate in 49 ml distilled water) is added. Twenty ml diluted citrate in 30 ml acetone under constant stirring) is added in a 6-well for 30sec. The cells are rinsed in distilled water and staining solution is added for 30 min at room temperature. For staining solution dissolve fast violet III capsule in 48 ml distilled water by stirring and add 2 ml Naphtol AS-Mix; filtrate solution. The cells are rinsed with distilled water for 2 min and kept wet. Pictures are taken with the Zeiss SteREO Lumar Microscope and the Zeiss Axio Vision IAC4.3 Software.

In Vivo

DBA/1 Mice

Male 8- to 12-wk-old DBA/1 mice are purchased from Janvier and housed following institutional guidelines. All animal procedures are approved by the institutional animal care and ethics committee. Mice are randomized and are, during a period of 8 days, treated daily with PBS (200 μl), DEX (20 μg or 62.5 μg dissolved in 200 μl PBS), PPARα agonist FF (200 mg/kg dissolved in 200 μl PBS) or DEX+PPARα agonist FF (20 μg or 62.5 μg for DEX and 200 mg/kg FF dissolved in 200 μl PBS). At day 8, murine serum is collected and used for the determination of TRAP5b and osteocalcin levels. The Mouse TRAP™ Assay is purchased from Immunodiagnostic Systems Ltd. The Mouse Osteocalcin EIA kit is purchased from Biomedical Technologies, Inc. All assays are performed according to the manufacturer's guidelines.

Statistical Analysis—All analyses are performed with the commercially available statistical Package GraphPad Prism 4. For normally distributed continuous data differences between groups are explored by one-way ANOVA, followed by a Dunnett's Multiple Comparison Test. If Gaussian distribution is not assumed, statistical significance is determined by means of the Kruskal-Wallis statistic, followed by a Dunn's Multiple Comparison Test.

Ex vivo: Pharmacological DEX concentrations inhibit osteoblastogenesis and inhibit relative expression of osteogenic marker genes. Treatment with a combination of DEX and a PPARα agonist will revert the osteoblastogenesis induction and will revert the inhibition of osteogenic merker genes.

In vivo: To examine the effect of the combination of PPARα agonists and GR ligands on osteoclast and osteoblast markers in vivo, DBA/1 mice are treated daily with solvent, DEX (20 μg), DEX (62.5 μg), PPARα agonist (fenofibrate at 4 mg) or combinations of DEX and PPARα agonist, e.g. DEX (20 μg)+4 mg fenofibrate or DEX (62.5 μg)+4 mg fenofibrate, during a time course of 8 days, after which mice serum is collected. A TRAP5b ELISA assay is performed for the detection of differentiated osteoclasts. DEX administration alone upregulates TRAP5b levels after 8 days. Additionally, DEX treatment significantly lowers the amount of osteocalcin in murine serum. Treatment with a combination of DEX and a PPARα agonist, administered together or separately is believed to prevent or reverse upregulation of TRAP5b levels and prevent or reverse the decrease in osteocalcin which results from glucocorticoid treatment.

### Example 26

The long-term effect of a combination of a PPARα agonist and a glucocorticoid agonist (synthetic glucocorticoid, DEX) on the glucocorticoid receptor-mediated transcriptional regulation of bone resorption genes, quantitative PCR analysis is performed in osteosarcoma cells.

Cell culture—Human osteosarcoma cells MG63b and Saos-2 are cultured in Dulbecco Modified Eagle's Medium (DMEM) and McCoy's 5a Medium respectively, supplemented with 10% fetal calf serum (FCS), 100 units/ml penicillin and 0.1 mg/ml streptomycin.

RT-PCR—After the appropriate inductions RNA is isolated from the cells by means of the RNeasy mini kit (Qiagen) according to the manufacturer's instructions. The mRNA is reverse transcribed with the verso cDNA kit (ABgene). The obtained cDNA is amplified by a quantitative PCR reaction with iQ Custom SYBR Green Supermix (Biorad). Gene expression of the housekeeping gene hypoxanthine-guanine phosphoribosyltransferase (HPRT) is used for normalization.

As glucocorticoids can influence gene expression both in a negative and in a positive manner, the effect of a 24 hour treatment protocol with DEX or DEX+PPARα agonist (e.g. WY at 10 μM) on the regulation of a glucocorticoid-upregulated gene involved in bone resorption, namely cathepsin K, is determined. With DEX at 10⁻⁶ M an upregulation of cathepsin K is expected in the human osteosarcoma cell line MG63b. WY at 10 μM is expected to prevent or reverse the upregulation of cathepsin K.

The effect of a combination of DEX and PPARα ligands on gene regulation of OPG is investigated. DEX at 10⁻⁶ M is expected to display a negative effect on the levels of OPG transcript in these cells.

Since the amount of free RANKL is an important marker for osteoclast differentiation, we are interested to investigate the effect of DEX and a combination of DEX and PPARα ligands on the RANKL/OPG ratio. As MG63b cells do not produce sufficient amounts of RANKL, for this purpose, the Saos-2 osteosarcoma cell line is used to determine the RANKL/OPG ratio. Treatment of the Saos-2 cells with DEX at 10-6 M for 24 hours is expected to result in a significant increase of RANKL expression. Upon calculating the ratio of RANK/OPG in Saos-2 DEX treatment is expected to evoke a rise in the RANKL/OPG ratio and treatment with a PPARα agonist will prevent or reverse the increase in RANKL/OPG ratio.

### Example 27

A human subject having insulin resistance as determined by impaired glucose tolerance is treated with 8 mg dexamethasone and 200 mg fenofibrate administered together orally once per day for 7 days. A 75 g oral glucose tolerance test is performed, measuring blood glucose levels at baseline and at 15, 30, 45, 60 and 90 minutes after glucose ingestion to demonstrate beneficial effects of the treatment on glucocorticoid-induced hyperglycemia. Impaired glucose tolerance in a human is well-defined, for example, as 2-hour plasma glucose of greater than or equal to 7.8 mmol/L and a level of greater than or equal to 11.1 mmol/L indicative of insulin resistance in diabetes mellitus.

### Example 28

A human kidney transplant subject is treated with glucocorticoids according to a standard treatment regimen to inhibit transplant-related inflammation and rejection. A dose of 500 mg methylprednisone is administered intravenously on the day of the transplant procedure, 100-200 mg/day is administered orally on day 1 post-procedure and tapered to achieve 20-30 mg/day on days 5-28 post-procedure and further tapered to achieve 5-10 mg/day 3-6 months post-procedure.

Glucocorticoid-induced insulin resistance is treated in the subject during methylprednisone treatment using 200 mg fenofibrate administered orally once per day during methylprednisone treatment. A 75 g oral glucose tolerance test is performed, measuring blood glucose levels at baseline and at 15, 30, 45, 60 and 90 minutes after glucose ingestion to demonstrate beneficial effects of the treatment on glucocorticoid-induced hyperglycemia.

### Example 29

In vitro skin models are used to demonstrate the effects of PPARα agonists and PPARγ agonists on glucocorticoid-induced skin thinning. Skin models are generated using normal human fibroblasts and keratinocytes isolated from donors as described in N. N. Zoller et al. Toxicology in Vitro, 22:747-759, 2008. Glucocorticoids 0.25% prednicarbate, 0.1% mometasonfuroate, 0.1% methylprednisolonaceponate, and 0.064% betamethasondipropionate are applied to the skin models with or without 0.25, 0.5 or 1 μM GW647; 2, 5 or 10 μM WY; 25 μmol/L fenofibrate; and/or 5 or 10 μM rosiglitazone to achieve the benefits of treatment on reduction of skin thinning. Histological analysis is performed to assess the results of these treatments by morphological comparison of treated and control samples.

### Example 30

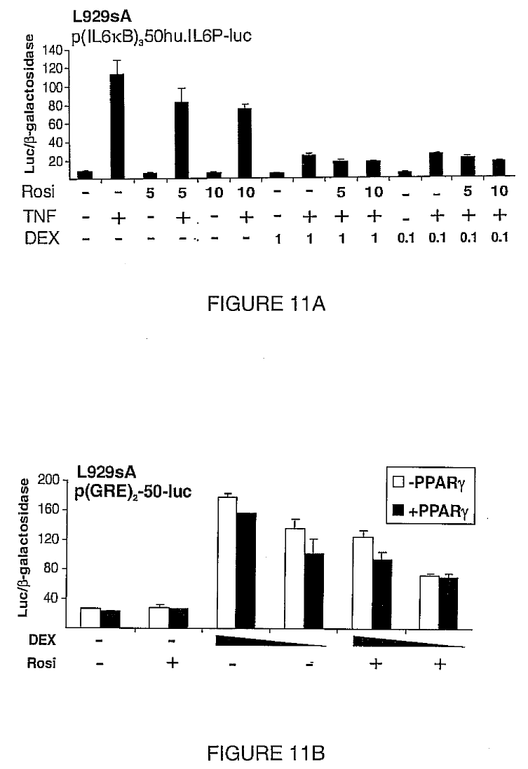

L929sA cells with stably integrated p(IL6 KB)₃50hu.IL6P-luc+ are pre-incubated with solvent, DEX (1 or 0.1 μM), rosiglitazone (Rosi) (5 or 10 μM) or various combinations thereof, as indicated in FIG. 11A, for 1 hr, before TNF (2000 IU/ml) is added, where indicated, for 6 h. Cell lysates are assayed for luc activities and normalized with β-gal activities. FIG. 11A shows results of this assay and indicates that the PPAPγ agonist Rosi blocks TNF-induced NF-κB-driven gene expression in a dose-responsive manner and that activated PPARγ cooperates with GRα to mediate an anti-inflammatory effect in addition to that achieved with DEX alone.

### Example 31

L929sA cells with stably integrated p(GRE)₂-50-luc are transiently transfected with either mock DNA or pSG5-PPARγ, upon which cells are pre-incubated, the day after transfection, with the appropriate solvent, DEX (1 μM or 0.1 μM), Rosi (10 μM), 0.1 μM DEX+10 μM Rosi, or 1 μM DEX+10 μM Rosi for 7 h. Cell lysates are assayed for luc activities and normalized with β-gal activities. FIG. 11B shows results of this assay and demonstrates antagonism between PPARγ and GRα.

### REFERENCES

1, De Bosscher, K., Vanden Berghe, W. & Haegeman, G. (2003) Endocr Rev 24, 488-522. The interplay between the glucocorticoid receptor and NF-κB or AP-1: molecular mechanisms for gene repression.
2. De Bosscher, K., Vanden Berghe, W. & Haegeman, G. (2006) Oncogene 25, 6868-86. Cross-talk between nuclear receptors and NF-κB.
3. Gilde, A. J., Fruchart, J. C. & Staels, B. (2006) J Am Coll Cardiol 48, A24-32. PPARs at the crossroads of obesity, diabetes, and cardiovascular disease.
4. Shiri-Sverdlov, R., Wouters, K., van Gorp, P. J., Gijbels, M. J., Noel, B., Buffat, L., Staels, B., Maeda, N., van Bilsen, M. & Hofker, M. H. (2006) J Hepatol 44, 732-41. Early diet-induced non-alcoholic steatohepatitis in APOE2 knock-in mice and its prevention by fibrates.
5. Desvergne, B. & Wahli, W. (1999) Endocr Rev 20, 649-88. PPARs: nuclear control of metabolism.
6. Delerive, P., De Bosscher, K., Besnard, S., Vanden Berghe, W., Peters, J. M., Gonzalez, F. J., Fruchart, J. C., Tedgui, A., Haegeman, G. & Staels, B. (1999) J Biol Chem 274, 32048-54. PPARα negatively regulates the vascular inflammatory gene response by negative cross-talk with transcription factors NF-κB and AP-1.
7. Staels, B., Koenig, W., Habib, A., Merval, R., Lebret, M., Torra, I. P., Delerive, P., Fadel, A., Chinetti, G., Fruchart, J. C., Najib, J., Maclouf, J. & Tedgui, A. (1998) Nature 393, 790-3. Activation of human aortic smooth-muscle cells is inhibited by PPARα but not by PPARγ activators.
8. Guerre-Millo, M., Gervois, P., Raspe, E., Madsen, L., Poulain, P., Derudas, B., Herbert, J. M., Winegar, D. A., Willson, T. M., Fruchart, J. C., Berge, R. K. & Staels, B. (2000) J Biol Chem 275, 16638-42. PPARα activators improve insulin sensitivity and reduce adiposity.
9. Nie, M., Corbett, L., Knox, A. J. & Pang, L. (2005) J Biol Chem 280, 2550-61. Differential regulation of chemokine expression by PPARγ agonists: interactions with glucocorticoids and beta2-agonists.
10. Cuzzocrea, S., Bruscoli, S., Mazzon, E., Crisafulli, C., Donato, V., Di Paola, R., Velardi, E., Esposito, E., Nocentini, G. & Riccardi, C. (2008) Mol Pharmacol 73, 323-337. PPARα contributes to the anti-inflammatory activity of glucocorticoids.
11. Kajimura, S., Seale, P., Tomaru, T., Erdjument-Bromage, H., Cooper, M. P., Ruas, J. L., Chin, S., Tempst, P., Lazar, M. A. & Spiegelman, B. M. (2008) Genes Dev 22, 1397-1409. Regulation of the brown and white fat programs through a PRDM16/CtBP transcriptional complex.
12. De Bosscher, K., Vanden Berghe, W., Beck, I. M., Van Molle, W., Hennuyer, N., Hapgood, J., Libert, C., Staels, B., Louw, A. & Haegeman, G. (2005) Proc Natl Acad Sci USA 102, 15827-32. A fully dissociated compound of plant origin for inflammatory gene repression.
13. Newton, R. & Holden, N. S. (2007) Mol Pharmacol 72, 799-809. Separating transrepression and transactivation: a distressing divorce for the glucocorticoid receptor*?
14. Bernal-Mizrachi, C., Weng, S., Feng, C., Finck, B. N., Knutsen, R. H., Leone, T. C., Coleman, T., Mecham, R. P., Kelly, D. P. & Semenkovich, C. F. (2003) Nat Med 9, 1069-75. Dexamethasone induction of hypertension and diabetes is PPARα dependent in LDL receptor-null mice.
15. Subramanian, S., DeRosa, M. A., Bernal-Mizrachi, C., Laffely, N., Cade, W. T., Yarasheski, K. E., Cryer, P. E. & Semenkovich, C. F. (2006) Am J Physiol Endocrinol Metab 291, E1365-71. PPARα activation elevates blood pressure and does not correct glucocorticoid-induced insulin resistance in humans.
16. Ogawa, S., Lozach, J., Benner, C., Pascual, G., Tangirala, R. K., Westin, S., Hoffmann, A., Subramaniam, S., David, M., Rosenfeld, M. G. & Glass, C. K (2005) Cell 122, 707-21. Molecular determinants of crosstalk between nuclear receptors and toll-like receptors.
17. Chinetti, G., Fruchart, J. C. & Staels, B. (2003) Curr Opin Lipidol 14, 459-68. PPARs: new targets for the pharmacological modulation of macrophage gene expression and function.
18. Berry, M. N. & Friend, D. S. (1969) J Cell Biol 43, 506-20. High-yield preparation of isolated rat liver parenchymal cells: a biochemical and fine structural study.
19. Vanden Berghe, W., Plaisance, S., Boone, E., De Bosscher, K., Schmitz, M. L., Fiers, W. & Haegeman, G. (1998) J Biol Chem 273, 3285-90. p38 and ERK MAPK pathways are required for NF-κB p65 transactivation mediated by TNF.
20. Wong, C., Rougier-Chapman, E. M., Frederick, J. P., Datto, M. B., Liberati, N. T., Li, J. M. & Wang, X. F. (1999) Mol Cell Biol 19, 1821-30. Smad3-Smad4 and AP-1 complexes synergize in transcriptional activation of the c-Jun promoter by TGFβ.
21. Adcock, I. M., Nasuhara, Y., Stevens, D. A. & Barnes, P. J. (1999) Br J Pharmacol 127, 1003-11. Ligand-induced differentiation of GR trans-repression and transactivation: preferential targetting of NF-κB and lack of IκB involvement.
22. Wang, J. C., Derynck, M. K., Nonaka, D. F., Khodabakhsh, D. B., Haqq, C. & Yamamoto, K. R. (2004) Proc Natl Acad Sci USA 101, 15603-8. Chromatin immunoprecipitation (ChIP) scanning identifies primary glucocorticoid receptor target genes.
Beato, M., P. Herrlich, and G. Schütz. 1995. Steroid hormone receptors: many actors in search of a plot. Cell. 83:851-857.
Boumpas, D. T., G. P. Chrousos, R. L. Wilder, T. R. Cupps, and J. E. Balow. 1993. Glucocorticoid therapy for immune-mediated diseases: basic and clinical correlates. Ann. Intern. Med. 119:1198-1208.
Hollenberg, S. M., and R. M. Evans. 1988. Multiple and cooperative trans-activation domains of the human glucocorticoid receptor. Cell. 55:899-906.
Mangelsdorf, D. J., C. Thummel, M. Beato, P. Herrlich, G. Schütz, K. Umesono, B. Blumberg, P. Kastner, M. Mark, P. Chambon, and R. M. Evans. 1995. The nuclear receptor superfamily: the second decade. Cell. 83:835-839.
Robinson-Rechavi, M., H. Escriva Garcia, and V. Laudet. 2003. The nuclear receptor superfamily. J Cell Sci. 116:585-586.
Pratt, W. B., Y. Morishima, M. Murphy, and M. Harrell. 2006. Chaperoning of glucocorticoid receptors. Handb. Exp. Pharmacol: 111-138.
Rosen, J., and J. N. Miner. 2005. The search for safer glucocorticoid receptor ligands. Endocr Rev. 26:452-464.
Wright, A. P., J. Zilliacus, I. J. McEwan, K. Dahlman-Wright, T. Almlof, J. Carlstedt-Duke, and J. A. Gustafsson. 1993. Structure and function of the glucocorticoid receptor. J. Steroid Biochem. Mol. Biol. 47:11-19.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of treating a glucocorticoid-responsive condition in a subject, comprising:
administering, in combination, a glucocorticoid receptor agonist and a peroxisome proliferator-activated receptor (PPAR) agonist in therapeutically effective amounts.

2. The method of claim 1 wherein the PPAR agonist is selected from the group consisting of: PPARα agonist, PPARγ agonist, PPARδ agonist, dual PPARα/γ agonist and pan PPAR agonist.

3. The method of claim 2 wherein the PPARα agonist is a fibrate.

4. The method of claim 2 wherein the PPARα agonist is selected from the group consisting of: beclofibrate, bezafibrate, ciprofibrate, clofibrate, etofibrate, fenofibrate, gemfibrozil, 2-methyl-2-(4-((4-methyl-2-(4-(trifluoromethyl)phenyl)thiazole-5-carboxamido)methyl)phenoxy)propanoic acid; 2-methyl-2-[[4-[2-[[(cyclohexylamino)carbonyl](4-cyclohexylbutyl)amino]ethyl]phenyl]thio]-propanoic acid; 2-[[4-[2-[[[(2,4-difluorophenyl)amino]carbonyl]heptylamino]ethyl]phenyl]thio]-2-methyl-propanoic acid; [[4-chloro-6-[(2,3-dimethylphenyl)amino]-2-pyrimidinyl]thio]-acetic acid; 2-methyl-2-(4-{3-[1-(4-methylbenzyl)-5-oxo-4,5-dihydro-1H-1,2,4-triazol-3-yl]propyl}phenoxy)propanoic acid; and 2-(4-(2-(1-Cyclohexanebutyl-3-cyclohexylureido)ethyl)phenylthio)-2-methylpropionic acid.

5. The method of claim 1 wherein the glucocorticoid receptor agonist is selected from the group consisting of: alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, medrysone, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide and ulobetasol.

6. The method of claim 1, wherein the amount of the glucocorticoid receptor agonist is less than an amount of the glucocorticoid receptor agonist necessary to achieve a therapeutic effect if administered in the absence of the PPAR agonist.

7. The method of claim 1, wherein the amount of the PPAR agonist is sufficient to reduce a side-effect of administration of the glucocorticoid receptor agonist.

8. A composition, comprising:
a glucocorticoid receptor agonist, a PPAR agonist and a pharmaceutically acceptable carrier, the glucocorticoid receptor agonist and the PPAR agonist each present in an amount which, in combination, is a therapeutically effective amount for treating a glucocorticoid-responsive condition in a subject.

9. The composition of claim 8, wherein the amount of the glucocorticoid receptor agonist is less than an amount of the glucocorticoid receptor agonist necessary to achieve a therapeutic effect if administered without the PPAR agonist.

10. The composition of claim 8, wherein the amount of the PPAR agonist is sufficient to reduce a side-effect of administration of the glucocorticoid receptor agonist.

11. The composition of claim 8, wherein the PPAR agonist is selected from the group consisting of: PPARα agonist, PPARγ agonist, PPARδ agonist, dual PPARα/γ agonist and pan PPAR agonist.

12. The composition of claim 11 wherein the PPARα agonist is a fibrate.

13. The composition of claim 11 wherein the PPARα agonist is selected from the group consisting of: beclofibrate, bezafibrate, ciprofibrate, clofibrate, etofibrate, fenofibrate, gemfibrozil, 2-methyl-2-(4-((4-methyl-2-(4-(trifluoromethyl)phenyl)thiazole-5-carboxamido)methyl)phenoxy)propanoic acid; 2-methyl-2-[[4-[2-[[(cyclohexylamino)carbonyl](4-cyclohexylbutyl)amino]ethyl]phenyl]thio]-propanoic acid; 2-[[4-[2-[[[(2,4-difluorophenyl)amino]carbonyl]heptylamino]ethyl]phenyl]thio]-2-methyl-propanoic acid; [[4-chloro-6-[(2,3-dimethylphenyl)amino]-2-pyrimidinyl]thio]-acetic acid; 2-methyl-2-(4-{3-[1-(4-methylbenzyl)-5-oxo-4,5-dihydro-1H-1,2,4-triazol-3-yl]propyl}phenoxy)propanoic acid; and 2-(4-(2-(1-Cyclohexanebutyl-3-cyclohexylureido)ethyl)phenylthio)-2-methylpropionic acid.

14. The composition of claim 8 wherein the glucocorticoid receptor agonist is selected from the group consisting of: alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, medrysone, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide and ulobetasol.

15. A kit, comprising:
a therapeutic agent selected from the group consisting of: a glucocorticoid receptor agonist, a PPAR agonist, and a combination thereof; and
instructions for administering the glucocorticoid receptor agonist and the PPAR agonist for treatment of a glucocorticoid-responsive condition in a subject.

16. The kit of claim 15 wherein the PPAR agonist is selected from the group consisting of: PPARα agonist, PPARγ agonist, PPARδ agonist, dual PPARα/γ agonist and pan PPAR agonist.

17. The kit of claim 16 wherein the PPARα agonist is selected from the group consisting of: beclofibrate, bezafibrate, ciprofibrate, clofibrate, etofibrate, fenofibrate, gemfibrozil, 2-methyl-2-(4-((4-methyl-2-(4-(trifluoromethyl)phenyl)thiazole-5-carboxamido)methyl)phenoxy)propanoic acid; 2-methyl-2-[[4-[2-[[(cyclohexylamino)carbonyl](4-cyclohexylbutyl)amino]ethyl]phenyl]thio]-propanoic acid; 2-[[4-[2-[[[(2,4-difluorophenyl)amino]carbonyl]heptylamino]ethyl]phenyl]thio]-2-methyl-propanoic acid; [[4-chloro-6-[(2,3-dimethylphenyl)amino]-2-pyrimidinyl]thio]-acetic acid; 2-methyl-2-(4-{3-[1-(4-methylbenzyl)-5-oxo-4,5-dihydro-1H-1,2,4-triazol-3-yl]propyl}phenoxy)propanoic acid; and 2-(4-(2-(1-Cyclohexanebutyl-3-cyclohexylureido)ethyl)phenylthio)-2-methylpropionic acid.

18. The kit of claim 15 wherein the glucocorticoid receptor agonist is selected from the group consisting of: alclometasone, alclometasone dipropionate, amcinonide, beclometasone, beclomethasone dipropionate, betamethasone, betamethasone benzoate, betamethasone valerate, budesonide, ciclesonide, clobetasol, clobetasol butyrate, clobetasol propionate, clobetasone, clocortolone, cloprednol, cortisol, cortisone, cortivazol, deflazacort, desonide, desoximetasone, desoxycortone, desoxymethasone, dexamethasone, diflorasone, diflorasone diacetate, diflucortolone, diflucortolone valerate, difluorocortolone, difluprednate, fluclorolone, fluclorolone acetonide, fludroxycortide, flumetasone, flumethasone, flumethasone pivalate, flunisolide, flunisolide hemihydrate, fluocinolone, fluocinolone acetonide, fluocinonide, fluocortin, fluocoritin butyl, fluocortolone, fluorocortisone, fluorometholone, fluperolone, fluprednidene, fluprednidene acetate, fluprednisolone, fluticasone, fluticasone propionate, formocortal, halcinonide, halometasone, hydrocortisone, hydrocortisone acetate, hydrocortisone aceponate, hydrocortisone buteprate, hydrocortisone butyrate, loteprednol, medrysone, meprednisone, 6a-methylprednisolone, methylprednisolone, methylprednisolone acetate, methylprednisolone aceponate, mometasone, mometasone furoate, mometasone furoate monohydrate, paramethasone, prednicarbate, prednisolone, prednisone, prednylidene, rimexolone, tixocortol, triamcinolone, triamcinolone acetonide and ulobetasol.

19. A method of treating insulin resistance in a subject, comprising:
administering, in combination, a glucocorticoid receptor agonist and a PPAR agonist in therapeutically effective amounts.

20. The method of claim 17 wherein the PPAR agonist is selected from the group consisting of: PPARα agonist, PPARγ agonist, PPARδ agonist, dual PPARα/γ agonist and pan PPAR agonist.

21. The method of claim 19, wherein the glucocorticoid receptor agonist is administered prior to the PPAR agonist.

22. The method of claim 19, wherein the glucocorticoid receptor agonist is administered substantially simultaneously with the PPAR agonist.


SEQUENCE LISTING (14 sequences)

SEQ ID NO: 1 - DNA, 23 bp, Artificial Sequence
  primer
gaggataccactcccaacagacc

SEQ ID NO: 2 - DNA, 24 bp, Artificial Sequence
  primer
aagtgcatcatcgttgttcataca

SEQ ID NO: 3 - DNA, 24 bp, Artificial Sequence
  primer
ccagtgtgctccagaaagtgtaag

SEQ ID NO: 4 - DNA, 24 bp, Artificial Sequence
  primer
agaaggctcatttggctcaatctc

SEQ ID NO: 5 - DNA, 20 bp, Artificial Sequence
  primer
gcgtgagaacaccctgttga

SEQ ID NO: 6 - DNA, 23 bp, Artificial Sequence
  primer
tcagacaggactggaacttctcc

SEQ ID NO: 7 - DNA, 20 bp, Artificial Sequence
  primer
tgccagcctcatgtattgga

SEQ ID NO: 8 - DNA, 20 bp, Artificial Sequence
  primer
ttcctggtccatcaacctgg

SEQ ID NO: 9 - DNA, 19 bp, Artificial Sequence
  primer
gccaactctcactgaagcc

SEQ ID NO: 10 - DNA, 20 bp, Artificial Sequence
  primer
gctggtgaatgagtagcagc

SEQ ID NO: 11 - DNA, 19 bp, Artificial Sequence
  primer
tgcccatttcgacgacgac

SEQ ID NO: 12 - DNA, 19 bp, Artificial Sequence
  primer
gtgcaggccgaataggagc

SEQ ID NO: 13 - DNA, 16 bp, Artificial Sequence
  primer
ggctgcaaggacatcg

SEQ ID NO: 14 - DNA, 17 bp, Artificial Sequence
  primer
cagttcagtgcggttcc